SEQUENCE LISTING (15 sequences)

SEQ ID NO: 1 - protein, 233 aa, Homo sapiens
MSQSNRELVVDFLSYKLSQKGYSWSQFSDVEENRTEAPEGTESEMETPSAINGNPSWHLADSPAVNGATGHSSSLDAREVIPMAAVKQALREAGDEFELRYRRAFSDLTSQLHITPGTAYQSFEQVVNELFRDGVNWGRIVAFFSFGGALCVESVDKEMQVLVSRIAAWMATYLNDHLEPWIQENGGWDTFVELYGNNAAAESRKGQERFNRWFLTGMTVAGVVLLGSLFSRK

SEQ ID NO: 2 - protein, 254 aa, Artificial Sequence
  Description of Artificial Sequence Synthetic polypeptide
MSQSNRELVVDFLSYKLSQKGYSWSQFSDVEENRTEAPEGTESEMETPSAINGNPSWHLADSPAVNGATGHSSSLDAREVIPMAAVKQALREAGDEFELRYRRAFSDLTSQLHITPGTAYQSFEQVVNELFRDGVNWGRIVAFFSFGGALCVESVDKEMQVLVSRIAAWMATYLNDHLEPWIQENGGWDTFVELYGNNAAAESRKGQERFNRWFLTGMTVAGVVLLGSLFSRKGSGATNFSLLKQAGDVEENPG

SEQ ID NO: 3 - protein, 360 aa, Homo sapiens
MAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 4 - protein, 380 aa, Artificial Sequence
  Description of Artificial Sequence Synthetic polypeptide
MAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSNGSGEGRGSLLTCGDVEENPG

SEQ ID NO: 5 - protein, 413 aa, Homo sapiens
MRQPPGESDMAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSELMPPGSCMPEEPKPKRGRRSWPRKRTATHTCDYAGCGKTYTKSSHLKAHRSDHLALHMKRHF

SEQ ID NO: 6 - protein, 493 aa, Artificial Sequence
  Description of Artificial Sequence Synthetic polypeptide
PMAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHFGSGQCTNYALLKLAGDVESNPG

SEQ ID NO: 7 - protein, 317 aa, Homo sapiens
MYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHM

SEQ ID NO: 8 - protein, 318 aa, Artificial Sequence
  Description of Artificial Sequence Synthetic polypeptide
PMYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHM

SEQ ID NO: 9 - protein, 439 aa, Homo sapiens
MPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 10 - protein, 440 aa, Artificial Sequence
  Description of Artificial Sequence Synthetic polypeptide
PMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 11 - protein, 620 aa, Homo sapiens
MAEARTSLSAHCRGPLATGLHPDLDLPGRSLATPAPSCYLLGSEPSSGLGLQPETHLPEGSLKRCCVLGLPPTSPASSSPCASSDVTSIIRSSQTSLVTCVNGLRSPPLTGDLGGPSKRARPGPASTDSHEGSLQLEACRKASFLKQEPADEFSELFGPHQQGLPPPYPLSQLPPGPSLGGLGLGLAGRVVAGRQACRWVDCCAAYEQQEELVRHIEKSHIDQRKGEDFTCFWAGCVRRYKPFNARYKLLIHMRVHSGEKPNKCMFEGCSKAFSRLENLKIHLRSHTGEKPYLCQHPGCQKAFSNSSDRAKHQRTHLDTKPYACQIPGCSKRYTDPSSLRKHVKAHSAKEQQVRKKLHAGPDTEADVLTECLVLQQLHTSTQLAASDGKGGCGLGQELLPGVYPGSITPHNGLASGLLPPAHDVPSRHHPLDATTSSHHHLSPLPMAESTRDGLGPGLLSPIVSPLKGLGPPPLPPSSQSHSPGGQPFPTLPSKPSYPPFQSPPPPPLPSPQGYQGSFHSIQSCFPYGDCYRMAEPAAGGDGLVGETHGFNPLRPNGYHSLSTPLPATGYEALAEASCPTALPQQPSEDVVSSGPEDCGFFPNGAFDHCLGHIPSIYTDT

SEQ ID NO: 12 - protein, 305 aa, Homo sapiens
MSVDPACPQSLPCFEASDCKESSPMPVICGPEENYPSLQMSSAEMPHTETVSPLPSSMDLLIQDSPDSSTSPKGKQPTSAEKSVAKKEDKVPVKKQKTRTVFSSTQLCVLNDRFQRQKYLSLQQMQELSNILNLSYKQVKTWFQNQRMKSKRWQKNNWPKNSNGVTQKASAPTYPSLYSSYHQGCLVNPTGNLPMWSNQTWNNSTWSNQTQNIQSWSNHSWNTQTWCTQSWNNQAWNSPFYNCGEESLQSCMQFQPNSPASDLEAALEAAGEGLNVIQQTTRYFSTPQTMDLFLNYSMNMQPEDV

SEQ ID NO: 13 - protein, 258 aa, Homo sapiens
MRSFNQVSSAPGGASKGGGEEPGKLPEPAEEESQVLRGTGHCKWFNVRMGFGFISMINREGSPLDIPVDVFVHQSKLFMEGFRSLKEGEPVEFTFKKSSKGLESIRVTGPGGSPCLGSERRPKGKTLQKRKPKGDRCYNCGGLDHHAKECSLPPQPKKCHYCQSIMHMVANCPHKNVAQPPASSQGRQEAESQPCTSTLPREVGGGHGCTSPPFPQEARAEISERSGRSPQEASSTKSSIAPEEQSKKGPSVQKRKKT

SEQ ID NO: 14 - DNA, 7675 bp, Venezuelan equine encephalitis virus
gaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgctggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagtctagcatatgggcgcgtgaattcgccgcgaattggcaagctgcttacatagaactcgcggcgattggcatgccgccttaaaatttttattttatttttcttttcttttccgaatcggattttgtttttaatatttcaaaaaaaaaaaaaaaaaaaaaaaaaa

SEQ ID NO: 15 - DNA, 7675 bp, Artificial Sequence
  Description of Artificial Sequence Synthetic polynucleotide
gaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaattcttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagtctagcatatgggcgcgtgaattcgccgcgaattggcaagctgcttacatagaactcgcggcgattggcatgccgccttaaaatttttattttatttttcttttcttttccgaatcggattttgtttttaatatttcaaaaaaaaaaaaaaaaaaaaaaaaaa

(19) United States
(12) Patent Application Publication
(10) Pub. No.: US 2022/0306991 A1
(43) Pub. Date: Sep. 29, 2022

(54) METHODS FOR OBTAINING INDUCED PLURIPOTENT STEM CELLS

(71) Applicant: BlueRock Therapeutics LP, Cambridge, MA (US)

(72) Inventors: Peter D. Tonge, Toronto (CA); Borko Tanasijevic, Hamilton (CA)

(73) Assignee: BlueRock Therapeutics LP, Cambridge, MA (US)

(21) Appl. No.: 17/704,922

(22) Filed: Mar. 25, 2022

Related U.S. Application Data
(60) Provisional application No. 63/166,071, filed on Mar. 25, 2021.

(51) Int. Cl. C12N 5/0789; C12N 15/867

(52) U.S. Cl. CPC C12N 5/0647; C12N 15/867; C12N 2501/125; C12N 2501/155; C12N 2501/2303; C12N 2501/606; C12N 2501/14; C12N 2501/602; C12N 2501/603; C12N 2501/604; C12N 2501/608; C12N 2501/605; C12N 2506/11; C12N 2799/027

(57) ABSTRACT

Provided herein are methods of obtaining induced pluripotent stem cells from cells of a hematopoietic lineage.

### CROSS-REFERENCE TO RELATED APPLICATION

This application claims priority from U.S. Provisional Application 63/166,071, filed Mar. 25, 2021, the disclosure of which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Mar. 23, 2022, is named 025450_US014_SL.txt and is 62,837 bytes in size.

### BACKGROUND OF THE INVENTION

Cell therapy provides great promise for the treatment of a variety of diseases and conditions. In cell therapy, autologous or allogeneic cells are transplanted into a patient to replace or repair defective or damaged tissue or cells that may have arisen from a multitude of medical conditions including genetic disorders, cancer, neurologic disorders, cardiac disorders, or eye-related issues. Pluripotent stem cells are especially useful for cell therapy, including pluripotent stem cells generated from somatic cells. The seminal work of K. Takahashi and S. Yamanaka demonstrated the induction of pluripotent stem cells from mouse fibroblasts transduced with retroviral vectors expressing four reprogramming transcription factors, Oct3/4, Klf4, Sox2, and c-Myc (Cell (2006) 126:663-76). However, retroviral vectors can cause insertional mutations in the host genome and thus are not ideal vectors to be used in clinical settings.

RNA-based approaches have thus been attempted for introducing reprogramming factors into somatic cells. One such approach utilizes alphavirus-based virus RNA replicons. Alphaviruses, which constitute a genus of more than 30 viruses in the Togaviridae family, are lipid-enveloped, positive-sense RNA viruses. New World alphaviruses include Eastern, Western, and Venezuelan equine encephalitis viruses (EEEV, WEEV, and VEEV, respectively) and are found in North and South Americas. Old World alphaviruses include chikungunya (CHIK), Sindbis, Ross River, and O'nyong-nyong viruses.

Alphaviruses contain a positive-sense single-stranded RNA genome approximately 14 kb in length. After entry into a host cell, the alphavirus particle undergoes disassembly and releases the genomic RNA into the cytoplasm of the cell. Translation of the viral genome yields a nonstructural polyprotein, P1234, which is subsequently cleaved by proteases to generate nonstructural proteins (nsP1, nsP2, nsP3, and nsP4). The nonstructural proteins are involved in RNA replication and transcription. A subgenomic RNA—26S RNA—is also produced from the viral genome through transcription. Translation of the 26S RNA produces a structural polyprotein, which is cleaved to generate structural proteins (e.g., C, E3, E2, 6K, and E1 for VEEV). The structural proteins are involved in budding and viral encapsidation. See, e.g., Shin et al., PNAS (2012) 109(41):16534-9; Jose et al., Future Microbiol. (2009) 4:837-56; Hardy and Strauss, J Virol. (1989) 63(11):4653-64; Melancon and Garoff, J Virol. (1987) 61(5):1301-9; and Strauss et al., Virology (1984) 133(1):92-110); and Glanville et al., PNAS (1976) 73(9):3059-63).

Alphavirus replicons do not involve a DNA intermediate for replication and thus provide a safer alternative to several other commonly used viral vectors including retroviral vectors (Yoshioka et al., Cell Stem Cell. (2013) 13(2):246-54; Yoshioka and Dowdy, PLOS ONE (2017) 12:e0182018). Alphaviruses, and VEE in particular, have been explored as vectors to carry genes encoding exogenous transcription factors in reprogramming somatic cells into induced pluripotent stem cells (iPSC). However, this approach has been attempted only in fibroblasts and blood outgrowth endothelial cells (BOECs). Neither cell type is particularly attractive clinically. Autologous fibroblasts are obtained from skin puncture of patients, which is invasive and painful. BOECs, though derived from peripheral blood, are rare cells and require a laborious and time-consuming process to establish.

Another integration-free approach to reprogramming utilizes episomal DNA plasmid vectors. Wen et al. used this approach to reprogram peripheral blood mononuclear cells into iPSCs (Stem Cell Rep. (2016) 6:873-84). But that approach requires careful calibration of the levels of various reprogramming factors introduced through multiple vectors.

Sendai viral vectors also have been used to carry genes encoding reprogramming factors. The Sendai vectors are negative-stranded Paramyxoviruses; the vector must be packaged into a virion. This approach is more complicated. It involves packaging cell lines and may introduce adventitious agents to the vector product.

There is therefore a need for an efficient and safe approach to obtaining iPSCs from peripheral blood cells.

### SUMMARY OF THE INVENTION

The present disclosure provides a method of obtaining a population of induced pluripotent stem cells (iPSCs) from starting cells of a hematopoietic lineage. The method comprises: introducing to the starting cells an alphavirus RNA expression construct encoding BCL-xL and one or more additional reprogramming factors selected from an OCT family protein, a KLF family protein, a MYC family protein, a SOX family protein, a LIN28 protein, a NANOG protein, and a p53 dominant negative protein, and culturing the starting cells to allow the expression of BCL-xL and the one or more additional reprogramming factors, thereby inducing the starting cells and their progeny to reprogram into iPSCs.

In one aspect, the present disclosure provides a population of induced pluripotent stem cells (iPSCs) obtained from starting cells of a hematopoietic lineage that are transfected with an alphavirus RNA expression construct encoding BCL-xL and one or more additional reprogramming factors selected from an Oct family protein, a KLF family protein, a Myc family protein, a SOX family protein, a LIN28 protein, a NANOG protein, and a p53 dominant negative protein.

The starting cells may be, for example, hematopoietic stem cells, erythroid progenitor cells, lymphoid progenitor cells, peripheral blood mononuclear cells, T lymphocytes, B lymphocytes, macrophages, monocytes, neutrophils, eosinophils, or dendritic cells of human origin. In some embodiments, the starting cells are erythroid progenitor cells obtained by culturing peripheral blood mononuclear cells (PBMCs) in the presence of erythropoietin (EPO), stem cell factor (SCF), and IL-3, optionally for five to ten or six to seven days. In further embodiments, the PBMCs are cultured in the presence of 0.5-5 IU/ml EPO, 50-200 ng/mL SCF, and 1-10 ng/mL IL-3.

In some embodiments, the RNA expression construct is introduced to the starting cells through electroporation. In further embodiments, the starting cells are cultured with a B18R protein prior to electroporation.

In another aspect, the present disclosure provides an alphavirus RNA expression construct encoding BCL-xL and one or more additional reprogramming factors selected from an OCT family protein, a KLF family protein, a MYC family protein, a SOX family protein, a LIN28 protein, a NANOG protein, and a p53 dominant negative protein.

Also provided are a DNA vector comprising a coding sequence for the alphavirus RNA expression construct herein, and a host cell (e.g., a human cell) comprising the alphavirus RNA expression construct or the DNA vector herein.

In some embodiments, the alphavirus RNA expression construct is self-replicative and comprises genes for one or more nonstructural proteins sufficient to render the construct self-replicating (e.g., nsP1, nsP2, nsP3, and nsP4 genes). In further embodiments, the alphavirus RNA expression construct is a Venezuelan equine encephalitis virus (VEEV) RNA expression construct and comprises VEEV nsP1, nsP2, nsP3, and nsP4 genes. In certain embodiments, the VEEV RNA expression construct contains one or more (e.g., two or more, three or more, four or more, five or more, or six or more) mutations from the corresponding region(s) of wildtype VEEV genome.

In some embodiments, the OCT family protein is OCT4 (e.g., a human OCT4); the KLF family protein is KLF4 (e.g., a human KLF4); the SOX family protein is SOX2 (e.g., a human SOX2); the LIN28 protein is LIN28B (e.g., a human LIN28B); and/or the MYC family protein is c-MYC (e.g., a human c-MYC).

In particular embodiments, the BCL-xL protein comprising SEQ ID NO:1 or an amino acid sequence at least 95% identical thereto; the OCT4 protein comprising SEQ ID NO:3 or an amino acid sequence at least 95% identical thereto; the KLF4 protein comprising SEQ ID NO:5 or an amino acid sequence at least 95% identical thereto; the SOX2 protein comprising SEQ ID NO:7 or an amino acid sequence at least 95% identical thereto; and/or the c-MYC protein comprising SEQ ID NO:9 or an amino acid sequence at least 95% identical thereto.

In some embodiments, the coding sequences for BCL-xL and the one or more additional reprogramming factors are separated a coding sequence for a 2A peptide or an internal ribosome entry site (IRES). In some embodiments, the coding sequences for BCL-xL and the one or more additional reprogramming factors are under the transcriptional control of a common promoter (e.g., a 26S promoter).

In some embodiments, the alphavirus RNA expression construct herein directs expression of an OCT family protein, a SOX family protein, BCL-xL, and a MYC family protein, and optionally a KLF family protein.

In another aspect, the present disclosure provides a method of obtaining a differentiated cell in vitro, comprising culturing the iPSCs obtained herein in the presence of differentiation-promoting agents. Also provided are differentiated cells obtained by differentiated from the iPSCs. In some embodiments, a differentiated cell obtained herein is a human immune cell, optionally selected from a T cell, a T cell expressing a chimeric antigen receptor (CAR), a suppressive T cell, a myeloid cell, a dendritic cell, and an immunosuppressive macrophage; a cell in the human nervous system, optionally selected from dopaminergic neuron, a microglial cell, an oligodendrocyte, an astrocyte, a cortical neuron, a spinal or oculomotor neuron, an enteric neuron, a Placode-derived cell, a Schwann cell, and a trigeminal or sensory neuron; a cell in the human cardiovascular system, optionally selected from a cardiomyocyte, an endothelial cell, and a nodal cell; a cell in the human metabolic system, optionally selected from a hepatocyte, a cholangiocyte, and a pancreatic beta cell, or a cell in the human ocular system, optionally selected from a retinal pigment epithelial cell, a photoreceptor cone cell, a photoreceptor rod cell, a bipolar cell, a ganglion cell, immune cells, neural cells, cardiovascular cells, or cells in the metabolic system. In particular embodiments, the differentiated cell is of ectoderm lineage (e.g., a neuron). In other particular embodiments, the differentiated cell is of mesoderm lineage (e.g., a cardiomyocyte).

The present disclosure also provides a pharmaceutical composition comprising the differentiated cell obtained herein and a pharmaceutically acceptable carrier. The disclosure also provides a method of treating a patient in need thereof, comprising administering the pharmaceutical composition to the patient; use of the differentiated cell for the manufacture of a medicament for treating a patient in need thereof, and the differentiated cell or pharmaceutical composition for use in treating a patient in need thereof.

Other features, objects, and advantages of the invention are apparent in the detailed description that follows. It should be understood, however, that the detailed description, while indicating embodiments and aspects of the invention, is given by way of illustration only, not limitation. Various changes and modification within the scope of the invention will become apparent to those skilled in the art from the detailed description.

### BRIEF DESCRIPTION OF THE FIGURES

FIG. 1 is a schematic diagram illustrating seven exemplary VEEV RNA reprogramming constructs (OKS-iBM, OKS-iGM, OKS-iG, OSB, OS-iB, OS-iM, and OS-iBM). nsP1-4: coding sequences for non-structural proteins. OCT4: coding sequence for octamer-binding transcription factor 4. KLF4: coding sequence for Krüppel-like factor 4. SOX2: coding sequence for SRY-box transcription factor 2. BCL-xL: B-cell lymphoma-extra large. GLIS1: coding sequence for Glis family zinc finger 1. IRES: internal ribosomal entry site. Gray bar with asterisk: coding sequence for a 2A peptide. AAA: poly-A sequence.

FIGS. 2A-D are flow cytometry graphs showing the expression of pluripotency associated markers in four VEE-EP-iPSC lines, VEE-EP-iPSC-1, -2, -3 and -4, respectively. VEE-EP-iPSC-1, -2, -3 and -4 were generated by electroporation of erythroid progenitor (EP) cells respectively with OKS-iBM, OKS-iGM, OKS-iG, and an episomal control. The EBNA OriP episomal control contained the reprogramming factors OCT4, SOX2, KLF4, L-MYC, LIN28, and p53 dominant negative (Epi5™ Episomal iPSC Reprogramming Kit, Thermo Fisher; Okita et al., Nat Meth. (2011) 8:409-12). Flow cytometry was performed on cultured cells at passage eight.

FIG. 3 is a bar graph showing that the four VEE-EP-iPSC lines were differentiated for 16 days by first inducing the neurectoderm lineage and then maturing the progenitors to a neuronal fate. TH⁺FOXA2⁺ dopamine neurons were quantified by flow cytometry. TH: tyrosine hydroxylase. FOXA2: forkhead box protein A2.

FIG. 4 is bar graph showing that the four VEE-EP-iPSC lines were differentiated for 7 days towards cardiac lineages with stage-specific modulation of WNT signaling. Cardiomyocytes were quantified by flow cytometry for cardiac troponin (cTNT).

FIGS. 5A and 5B show the efficiency of VEE RNA reprogramming of EPs. EPs were electroporated with reprogramming constructs illustrated in FIG. 1. An episomal control as described above for FIG. 2 was used. FIG. 5A is a panel of photographs showing whole well imaging of TRA-1-60 staining. FIG. 5B is a graph quantifying TRA-1-60⁺ colonies.

FIG. 6A shows the nucleotide sequence (SEQ ID NO:14) of a wildtype VEEV RNA genomic sequence (except that T in the sequence is U for RNA).

FIG. 6B shows the nucleotide sequence (SEQ ID NO:15) of a recombinant VEEV RNA expression vector (except that T in the sequence is U for RNA). This sequence contains six mutations (C352G, A1564G, C1567A, T1570C, C1647A, and C3917T) relative to the wildtype sequence. The cloning site is indicated by an asterisk.

### DETAILED DESCRIPTION OF THE INVENTION

The present disclosure describes improved methods for reprogramming blood-derived cells (e.g., erythroid progenitors) into induced pluripotent stem cells (iPSCs). These methods involve the use of alphavirus (e.g., VEEV) RNA expression vectors (i.e., or expression constructs) encoding reprogramming factor BCL-xL and one or more (e.g., one, two, three, four, five, six, seven, or all eight) additional reprogramming factors (e.g., an OCT family member, a KLF family member, a SOX family member, a MYC protein, a NANOG protein, a GLIS family member, a LIN28 protein, and a p53 dominant negative). The alphavirus RNA expression constructs may be introduced to the blood cells through improved methods described herein. The transfected cells develop into harvestable iPSCs in less than 3 weeks.

Peripheral blood is a readily accessible cell source for the reprogramming of somatic cells to iPSCs. Thus, the present methods greatly improve the efficiency for generating iPSCs. Due to the use of an RNA-based expression vector that does not integrate into the host cells, the iPSCs obtained by the present methods have safer clinical profiles than those obtained by prior methods using retroviral vectors.

### I. Alphavirus RNA Constructs for Expressing Reprogramming Factors

The alphavirus RNA expression construct of the present disclosure is a self-replicating RNA replicon. A self-replicating RNA replicon or construct refers to an RNA molecule expressing nonstructural protein genes such that it can direct its own replication in a host cell. It may comprise 5′ and 3′ alphavirus replication recognition sequences, coding sequences for alphavirus nonstructural proteins that are essential for RNA replication and transcription (e.g., VEE nsP1, nsP2, nsP3, and nsP4), and a polyadenylation signal sequence. It may additionally contain one or more elements (e.g., IRES sequences, core or mini-promoters and the like) to direct the expression of a heterologous RNA sequence such as one coding for a reprogramming factor.

In some embodiments, the alphavirus RNA construct is a VEEV RNA replicon comprising (i) genes for VEEV non-structural proteins that are necessary for replication, (ii) 5′ and 3′ viral replication recognition sequences, (iii) expression cassette(s), such as a polycistronic expression cassette, for expressing reprogramming factors of interest; and (iv) a polyadenylation tail. See also Yoshioka 2013 and 2017, supra; and WO 2013/177133, and U.S. Pat. Nos. 10,793,833, 10,370,646, and 9,862,930. The replicon may lack VEEV structural proteins genes. A self-replicating VEE RNA construct can replicate inside transfected cells during a limited number of cell divisions. The timing of RNA construct loss by degradation can be further regulated by B18R withdrawal from the culture medium.

The exemplary VEEV RNA construct expresses BCL-xL and other reprogramming factors. A reprogramming factor is a protein that, when overexpressed in a somatic cell, induces a cell to transition from a differentiated state to a pluripotent state. The reprogramming factors used herein may be human proteins or modified versions thereof that retain the desired biological effects.

A. BCL-xL

Human BCL-xL is encoded by the BCL2L1 gene. An exemplary human BCL-xL amino acid sequence may be found at UniProt Accession No. Q07817 and has the following amino acid sequence:

(SEQ ID NO: 1)
MSQSNRELVV DFLSYKLSQK GYSWSQFSDV EENRTEAPEG
TESEMETPSA INGNPSWHLA DSPAVNGATG HSSSLDAREV
IPMAAVKQAL REAGDEFELR YRRAFSDLTS QLHITPGTAY
QSFEQVVNEL FRDGVNWGRI VAFFSFGGAL CVESVDKEMQ
VLVSRIAAWM ATYLNDHLEP WIQENGGWDT FVELYGNNAA
AESRKGQERF NRWFLTGMTV AGVVLLGSLF SRK

A functional analog of this protein, i.e., a molecule having the same or substantially the same biological function (e.g., retaining 70% or more, 80% or more, 90% or more, 95% or more, or 98% or more) of the protein's transcription factor function) is encompassed by the present disclosure as a BCL-xL protein. For example, the functional analog may be an isoform or a variant of the above protein, e.g., containing a portion of the above protein with or without additional amino acid residues and/or containing mutations relative to the above protein. In some embodiments, the functional analog has a sequence identity that is at least 90, 95, 98, or 99% to SEQ ID NO: 1. The percent identity of two amino acid sequences (or of two nucleic acid sequences) may be obtained by, e.g., BLAST® using default parameters (available at the U.S. National Library of Medicine's National Center for Biotechnology Information website). In some embodiments, the length of a reference sequence aligned for comparison purposes is at least 30%, (e.g., at least 40, 50, 60, 70, 80, or 90% of the reference sequence.

In certain embodiments, the BCL-xL protein expressed by the construct herein has the following sequence, wherein the residues in box are remnants from a 2A self-cleaving peptide after processing (a different self-cleaving peptide may leave different remnants or no remnant):

(SEQ ID NO: 2)
MSQSNRELVV DFLSYKLSQK GYSWSQFSDV EENRTEAPEG TESEMETPSA
INGNPSWHLA DSPAVNGATG HSSSLDAREV IPMAAVKQAL REAGDEFELR
YRRAFSDLTS QLHITPGTAY QSFEQVVNEL FRDGVNWGRI VAFFSFGGAL
CVESVDKEMQ VLVSRIAAWM ATYLNDHLEP WIQENGGWDT FVELYGNNAA

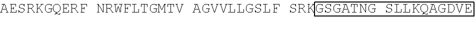

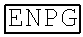

B. OCT Family Members

The exemplary VEEV construct may include a coding sequence for an Oct family protein (e.g., OCT1, OCT2, OCT4, OCT6, OCT7, OCT8, OCT9, and OCT11). See, e.g., U.S. Pat. No. 8,278,104 and WO 2013/177133. Human OCT4 is encoded by the POU5F1 gene. An exemplary human OCT4 amino acid sequence may be found at UniProt Accession No. Q01860 and has the following amino acid sequence:

(SEQ ID NO: 3)
MAGHLASDFA FSPPPGGGGD GPGGPEPGWV DPRTWLSFQG
PPGGPGIGPG VGPGSEVWGI PPCPPPYEFC GGMAYCGPQV
GVGLVPQGGL ETSQPEGEAG VGVESNSDGA SPEPCTVTPG
AVKLEKEKLE QNPEESQDIK ALQKELEQFA KLLKQKRITL
GYTQADVGLT LGVLFGKVFS QTTICRFEAL QLSFKNMCKL
RPLLQKWVEE ADNNENLQEI CKAETLVQAR KRKRTSIENR
VRGNLENLFL QCPKPTLQQI SHIAQQLGLE KDVVRVWFCN
RRQKGKRSSS DYAQREDFEA AGSPFSGGPV SFPLAPGPHF
GTPGYGSPHF TALYSSVPFP EGEAFPPVSV TTLGSPMHSN

A functional analog of this protein, i.e., a molecule having the same or substantially the same biological function (e.g., retaining 70% or more, 80% or more, 90% or more, 95% or more, or 98% or more of the protein's transcription factor function) is encompassed by the present disclosure as an OCT4 protein. For example, the functional analog may be an isoform or a variant of the above protein, e.g., containing a portion of the above protein with or without additional amino acid residues and/or containing mutations relative to the above protein. In some embodiments, the functional analog has a sequence identity that is at least 90, 95, 98, or 99% to SEQ ID NO:3.

In certain embodiments, the OCT4 protein expressed by the construct herein has the following sequence, wherein the residues in box are remnants from a 2A self-cleaving peptide after processing (a different self-cleaving peptide may leave different remnants or no remnant):

(SEQ ID NO: 4)
MAGHLASDFA FSPPPGGGGD GPGGPEPGWV DPRTWLSFQG PPGGPGIGPG
VGPGSEVWGI PPCPPPYEFC GGMAYCGPQV GVGLVPQGGL ETSQPEGEAG
VGVESNSDGA SPEPCTVTPG AVKLEKEKLE QNPEESQDIK ALQKELEQFA
KLLKQKRITL GYTQADVGLT LGVLFGKVFS QTTICRFEAL QLSFKNMCKL
RPLLQKWVEE ADNNENLQEI CKAETLVQAR KRKRTSIENR VRGNLENLFL
QCPKPTLQQI SHIAQQLGLE KDVVRVWFCN RRQKGKRSSS DYAQREDFEA
AGSPFSGGPV SFPLAPGPHF GTPGYGSPHF TALYSSVPFP EGEAFPPVSV

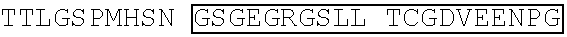

C. KLF Family Members

The exemplary VEEV construct may include a coding sequence for a KLF family protein (e.g., KLF1, KLF2, KLF3, KLF4, KLF5, KLF6, KLF7, KLF8, KLF9, KLF10, KLF11, KLF12, KLF13, KLF14, KLF15, KLF16, and KLF17). See, e.g., U.S. Pat. No. 8,278,104 and WO 2013/177133. Human KLF4 is encoded by the KLF4 gene. An exemplary human KLF4 amino acid sequence may be found at UniProt Accession No. 043474 and has the following amino acid sequence:

(SEQ ID NO: 5)
MRQPPGESDM AVSDALLPSF STFASGPAGR EKTLRQAGAP
NNRWREELSH MKRLPPVLPG RPYDLAAATV ATDLESGGAG
AACGGSNLAP LPRRETEEFN DLLDLDFILS NSLTHPPESV
AATVSSSASA SSSSSPSSSG PASAPSTCSF TYPIRAGNDP
GVAPGGTGGG LLYGRESAPP PTAPFNLADI NDVSPSGGFV
AELLRPELDP VYIPPQQPQP PGGGLMGKPV LKASLSAPGS
EYGSPSVISV SKGSPDGSHP VVVAPYNGGP PRTCPKIKQE
AVSSCTHLGA GPPLSNGHRP AAHDFPLGRQ LPSRTTPTLG
LEEVLSSRDC HPALPLPPGF HPHPGPNYPS ELMPPGSCMP
EEPKPKRGRR SWPRKRTATH TCDYAGCGKT YTKSSHLKAH
RSDHLALHMK RHF

A functional analog of this protein, i.e., a molecule having the same or substantially the same biological function (e.g., retaining 70% or more, 80% or more, 90% or more, 95% or more, or 98% or more of the protein's transcription factor function) is encompassed by the present disclosure as a KLF4 protein. For example, the functional analog may be an isoform or a variant of the above protein, e.g., containing a portion of the above protein with or without additional amino acid residues and/or containing mutations relative to the above protein. In some embodiments, the functional analog has a sequence identity that is at least 90, 95, 98, or 99% to SEQ ID NO:5. In some embodiments, ESSRB may be used in lieu of a KLF protein. In some embodiments, the KLF4 protein is an isoform of SEQ ID NO:5 and comprises amino acid residues 2-471 of SEQ ID NO:6 shown below.

In certain embodiments, the KLF4 protein expressed by the construct herein has the following sequence, wherein the residues in box are remnants from a 2A self-cleaving peptide after processing (a different self-cleaving peptide may leave different remnants or no remnant):

(SEQ ID NO: 6)

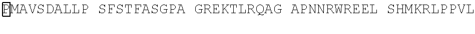

PGRPYDLAAA TVATDLESGG AGAACGGSNL APLPRRETEE FNDLLDLDFI
LSNSLTHPPE SVAATVSSSA SASSSSSPSS SGPASAPSTC SFTYPIRAGN
DPGVAPGGTG GGLLYGRESA PPPTAPFNLA DINDVSPSGG FVAELLRPEL
DPVYIPPQQP QPPGGGLMGK FVLKASLSAP GSEYGSPSVI SVSKGSPDGS
HPVVVAPYNG GPPRTCPKIK QEAVSSCTHL GAGPPLSNGH RPAAHDFPLG
RQLPSRTTPT LGLEEVLSSR DCHPALPLPP GFHPHPGPNY PSFLPDQMQP
QVPPLHYQEL MPPGSCMPEE PKPKRGRRSW PRKRTATHTC DYAGCGKTYT
KSSHLKAHLR THTGEKPYHC DWDGCGWKFA RSDELTRHYR KHTGHRPFQC

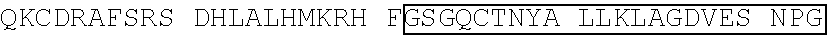

D. SOX Family Members

The exemplary VEEV construct may include a coding sequence for a SOX family protein (e.g., SOX1, SOX2, SOX3, SOX4, SOX5, SOX6, SOX7, SOX8, SOX9, SXO10, SOX11, SOX12, SOX13, SOX14, SOX15, SOX17, SOX18, SOX21, and SOX30). See, e.g., U.S. Pat. No. 8,278,104 and WO 2013/177133. Human SOX2 is encoded by the SOX2 gene. An exemplary human SOX2 amino acid sequence may be found at UniProt Accession No. P48431 and has the following amino acid sequence:

(SEQ ID NO: 7)
MYNMMETELK PPGPQQTSGG GGGNSTAAAA GGNQKNSPDR
VKRPMNAFMV WSRGQRRKMA QENPKMHNSE ISKRLGAEWK
LLSETEKRPF IDEAKRLRAL HMKEHPDYKY RPRRKTKTLM
KKDKYTLPGG LLAPGGNSMA SGVGVGAGLG AGVNQRMDSY
AHMNGWSNGS YSMMQDQLGY PQHPGLNAHG AAQMQPMHRY
DVSALQYNSM TSSQTYMNGS PTYSMSYSQQ GTPGMALGSM
GSVVKSEASS SPPVVTSSSH SRAPCQAGDL RDMISMYLPG
AEVPEPAAPS RLHMSQHYQS GPVPGTAING TLPLSHM

A functional analog of this protein, i.e., a molecule having the same or substantially the same biological function (e.g., retaining 70% or more, 80% or more, 90% or more, 95% or more, or 98% or more of the protein's transcription factor function) is encompassed by the present disclosure as a SOX2 protein. For example, the functional analog may be an isoform or a variant of the above protein, e.g., containing a portion of the above protein with or without additional amino acid residues and/or containing mutations relative to the above protein. In some embodiments, the functional analog has a sequence identity that is at least 90, 95, 98, or 99% to SEQ ID NO:7.

In certain embodiments, the SOX2 protein expressed by the construct herein has the following sequence, wherein the residue in box is a remnant from a 2A self-cleaving peptide after processing (a different self-cleaving peptide may leave different remnants or no remnant):

(SEQ ID NO: 8)

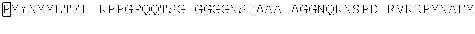

VWSRGQRRKM AQENPKMHNS EISKRLGAEW KLLSETEKRP FIDEAKRLRA
LHMKEHPDYK YRPRRKTKTL MKKDKYTLPG GLLAPGGNSM ASGVGVGAGL
GAGVNQRMDS YAHMNGWSNG SYSMMQDQLG YPQHPGLNAH GAAQMQPMHR
YDVSALQYNS MTSSQTYMNG SPTYSMSYSQ QGTPGMALGS MGSVVKSEAS
SSPPVVTSSS HSRAPCQAGD LRDMISMYLP GAEVPEPAAP SRLHMSQHYQ
SGPVPGTAIN GTLPLSHM

E. MYC Family Members

The exemplary VEEV construct may include a coding sequence for a MYC family protein (e.g., c-MYC, n-MYC, and 1-MYC). See, e.g., U.S. Pat. No. 8,278,104. Human c-MYC is encoded by the MYC gene. An exemplary human c-MYC amino acid sequence may be found at UniProt Accession No. P01106 and has the following amino acid sequence:

(SEQ ID NO: 9)
MPLNVSFTNR NYDLDYDSVQ PYFYCDEEEN FYQQQQQSEL
QPPAPSEDIW KKFELLPTPP LSPSRRSGLC SPSYVAVTPF
SLRGDNDGGG GSFSTADQLE MVTELLGGDM VNQSFICDPD
DETFIKNIII QDCMWSGFSA AAKLVSEKLA SYQAARKDSG
SPNPARGHSV CSTSSLYLQD LSAAASECID PSVVFPYPLN
DSSSPKSCAS QDSSAFSPSS DSLLSSTESS PQGSPEPLVL
HEETPPTTSS DSEEEQEDEE EIDVVSVEKR QAPGKRSESG
SPSAGGHSKP PHSPLVLKRC HVSTHQHNYA APPSTRKDYP
AAKRVKLDSV RVLRQISNNR KCTSPRSSDT EENVKRRTHN
VLERQRRNEL KRSFFALRDQ IPELENNEKA PKVVILKKAT
AYILSVQAEE QKLISEEDLL RKRREQLKHK LEQLRNSCA

A functional analog of this protein, i.e., a molecule having the same or substantially the same biological function (e.g., retaining 70% or more, 80% or more, 90% or more, 95% or more, or 98% or more of the protein's transcription factor function) is encompassed by the present disclosure as a c-MYC protein. For example, the functional analog may be an isoform or a variant of the above protein, e.g., containing a portion of the above protein with or without additional amino acid residues and/or containing mutations relative to the above protein. In some embodiments, the functional analog has a sequence identity that is at least 90, 95, 98, or 99% to SEQ ID NO:9. In some embodiments, a MYC variant having reduced transformation activity may be used in lieu of c-MYC. See, e.g., U.S. Pat. No. 9,005,967.

In certain embodiments, the c-MYC protein expressed by the construct herein has the following sequence, wherein the residue in box is a remnant from a 2A self-cleaving peptide after processing (a different self-cleaving peptide may leave different remnants or no remnant):

(SEQ ID NO: 10)

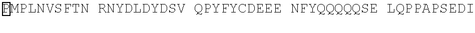

WKKFELLPTP PLSPSRRSGL CSPSYVAVTP FSLRGDNDGG GGSFSTADQL
EMVTELLGGD MVNQSFICDP DDETFIKNII IQDCMWSGFS AAAKLVSEKL
ASYQAARKDS GSPNPARGHS VCSTSSLYLQ DLSAAASECI DPSVVFPYPL
NDSSSPKSCA SQDSSAFSPS SDSLLSSTES SPQGSPEPLV LHEETPPTTS
SDSEEEQEDE EEIDVVSVEK RQAPGKRSES GSPSAGGHSK PPHSPLVLKR
CHVSTHQHNY AAPPSTRKDY PAAKRVKLDS VRVLRQISNN RKCTSPRSSD
TEENVKRRTH NVLERQRRNE LKRSFFALRD QIPELENNEK APKVVILKKA
TAYILSVQAE EQKLISEEDL LRKRREQLKH KLEQLRNSCA

F. GLIS Family Members

The exemplary VEEV construct may include a coding sequence for a GLIS family protein (e.g., GLIS1, GLIS2, and GLIS3). See, e.g., U.S. Pat. No. 8,951,801. Human GLIS1 is encoded by the GLIS1 gene. An exemplary human GLIS1 amino acid sequence may be found at UniProt Accession No. Q8NBF1 and has the following amino acid sequence:

(SEQ ID NO: 11)
MAEARTSLSA HCRGPLATGL HPDLDLPGRS LATPAPSCYL
LGSEPSSGLG LQPETHLPEG SLKRCCVLGL PPTSPASSSP
CASSDVTSII RSSQTSLVTC VNGLRSPPLT GDLGGPSKRA
RPGPASTDSH EGSLQLEACR KASFLKQEPA DEFSELFGPH
QQGLPPPYPL SQLPPGPSLG GLGLGLAGRV VAGRQACRWV
DCCAAYEQQE ELVRHIEKSH IDQRKGEDFT CFWAGCVRRY
KPFNARYKLL IHMRVHSGEK PNKCMFEGCS KAFSRLENLK
IHLRSHTGEK PYLCQHPGCQ KAFSNSSDRA KHQRTHLDTK
PYACQIPGCS KRYTDPSSLR KHVKAHSAKE QQVRKKLHAG
PDTEADVLTE CLVLQQLHTS TQLAASDGKG GCGLGQELLP
GVYPGSITPH NGLASGLLPP AHDVPSRHHP LDATTSSHHH
LSPLPMAEST RDGLGPGLLS PIVSPLKGLG PPPLPPSSQS
HSPGGQPFPT LPSKPSYPPF QSPPPPPLPS PQGYQGSFHS
IQSCFPYGDC YRMAEPAAGG DGLVGETHGF NPLRPNGYHS
LSTPLPATGY EALAEASCPT ALPQQPSEDV VSSGPEDCGF
FPNGAFDHCL GHIPSIYTDT

A functional analog of this protein, i.e., a molecule having the same or substantially the same biological function (e.g., retaining 70% or more, 80% or more, 90% or more, 95% or more, or 98% or more of the protein's transcription factor function) is encompassed by the present disclosure as a GLIS1 protein. For example, the functional analog may be an isoform or a variant of the above protein, e.g., containing a portion of the above protein with or without additional amino acid residues and/or containing mutations relative to the above protein. In some embodiments, the functional analog has a sequence identity that is at least 90, 95, 98, or 99% to SEQ ID NO:11.

G. NANOG

The present VEEV construct may include a coding sequence for NANOG. See, e.g., U.S. Pat. No. 9,506,039. Human NANOG is encoded by the NANOG gene. An exemplary human NANOG amino acid sequence may be found at UniProt Accession No. Q9H9S0 and has the following amino acid sequence:

(SEQ ID NO: 12)
MSVDPACPQS LPCFEASDCK ESSPMPVICG PEENYPSLQM
SSAEMPHTET VSPLPSSMDL LIQDSPDSST SPKGKQPTSA
EKSVAKKEDK VPVKKQKTRT VFSSTQLCVL NDRFQRQKYL
SLQQMQELSN ILNLSYKQVK TWFQNQRMKS KRWQKNNWPK
NSNGVTQKAS APTYPSLYSS YHQGCLVNPT GNLPMWSNQT
WNNSTWSNQT QNIQSWSNHS WNTQTWCTQS WNNQAWNSPF
YNCGEESLQS CMQFQPNSPA SDLEAALEAA GEGLNVIQQT
TRYFSTPQTM DLFLNYSMNM QPEDV

A functional analog of this sequence, i.e., a molecule having the same or substantially the same biological function (e.g., retaining 70% or more, 80% or more, 90% or more, 95% or more, or 98% or more of the protein's transcription factor function) of the above protein is encompassed by the present disclosure as NANOG protein. For example, the functional analog may be an isoform or a variant of the above protein, e.g., containing a portion of the above protein with or without additional amino acid residues and/or containing mutations relative to the above protein. In some embodiments, the functional analog has a sequence identity that is at least 90, 95, 98, or 99% to SEQ ID NO:12.

H. LIN28 Proteins

The present VEEV construct may include a coding sequence for a LIN28 protein (e.g., LIN28A or LIN28B). See, e.g., U.S. Pat. No. 9,506,039. Human LIN28B is encoded by the LIN28B gene. An exemplary human LIN28B amino acid sequence may be found at UniProt Accession No. A0A1B0GVD3 and has the following amino acid sequence:

(SEQ ID NO: 13)
MRSFNQVSSA PGGASKGGGE EPGKLPEPAE EESQVLRGTG
HCKWFNVRMG FGFISMINRE GSPLDIPVDV FVHQSKLFME
GFRSLKEGEP VEFTFKKSSK GLESIRVTGP GGSPCLGSER
RPKGKTLQKR KPKGDRCYNC GGLDHHAKEC SLPPQPKKCH
YCQSIMHMVA NCPHKNVAQP PASSQGRQEA ESQPCTSTLP
REVGGGHGCT SPPFPQEARA EISERSGRSP QEASSTKSSI
APEEQSKKGP SVQKRKKT

A functional analog of this sequence, i.e., a molecule having the same or substantially the same biological function (e.g., retaining 70% or more, 80% or more, 90% or more, 95% or more, or 98% or more of the protein's transcription factor function) of the above protein is encompassed by the present disclosure as a LIN28B protein. For example, the functional analog may be an isoform or a variant of the above protein, e.g., containing a portion of the above protein with or without additional amino acid residues and/or containing mutations relative to the above protein. In some embodiments, the functional analog has a sequence identity that is at least 90, 95, 98, or 99% to SEQ ID NO:13.

Exemplary functional analogs of the reprogramming factors described herein are described in, e.g., Yang et al., Asian J Andrology (2015) 17:394-402, the disclosure of which is incorporated herein by reference in its entirety.

I. RNA Expression Constructs

In some embodiments, the coding sequences of the reprogramming factors might be incorporated into one or more expression cassettes, each having its own promoter (e.g., a 26S promoter) and other transcription regulatory elements.

In some embodiments, the coding sequences of the reprogramming factors may be placed in frame in a polycistronic expression cassette such that they are transcribed from a common promoter (e.g., a 26S or SP6 promoter). These coding sequences may be separated by translation-skipping sequences (i.e., in-frame coding sequences for a self-cleaving peptide), such that translation of the mRNA transcript from the polycistronic cassette will result in separate proteins. A self-cleaving peptide causes ribosomal skipping during translation. Examples of self-cleaving peptides are 2A peptides, which are viral derived peptides with a typical length of 18-22 amino acids. 2A peptides include T2A, P2A, E2A, F2A, and PQR (Lo et al., Cell Reports (2015) 13:2634-2644). By way of example, P2A is a peptide of 19 amino acids; after the cleavage, a few amino acid residues from the P2A are left on the upstream gene and a proline is left at the beginning of the second gene. The coding sequences for the reprogramming factors also may be separated instead by an internal ribosome entry site (IRES) in the mRNA. IRES also allows for translation of separate polypeptides from a common RNA transcript. 2A residues left on the processed polypeptides do not affect the functionality of the polypeptides.

By way of example, the alphavirus RNA construct may comprise from 5′ to 3′: [alphavirus 5′ UTR]-[genes for alphavirus RNA replicases]-[promoter]-[reprogramming factor 1 coding sequence]-[2A peptide coding sequence]-[reprogramming factor 2 coding sequence]-[2A peptide coding sequence]-[reprogramming factor 3 coding sequence]-[IRES or core promoter]-[reprogramming factor 4 coding sequence]-[2A peptide coding sequence]-[reprogramming factor 5]-[optional selectable marker]-[alphavirus 3′ UTR and poly A tail]. The poly A tail length may vary (e.g., from 10 to more than 200 adenosines), and the order of the reprogramming factors may change without affecting the reprogramming function of the RNA construct. The promoter for the polycistronic reprogramming factor expression cassette may be, for example, a 26S internal promoter. In some embodiments, the alphavirus RNA construct is a VEEV RNA construct and the genes for its replicase is VEEV RNA replicase 1, 2, 3, and 4.

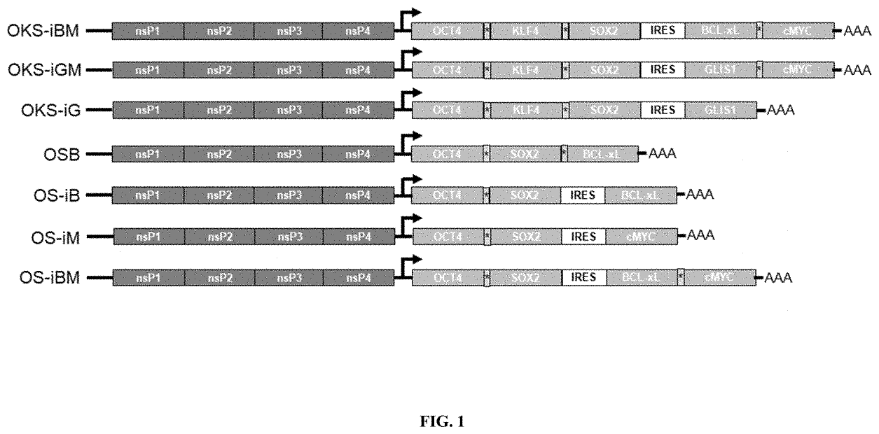

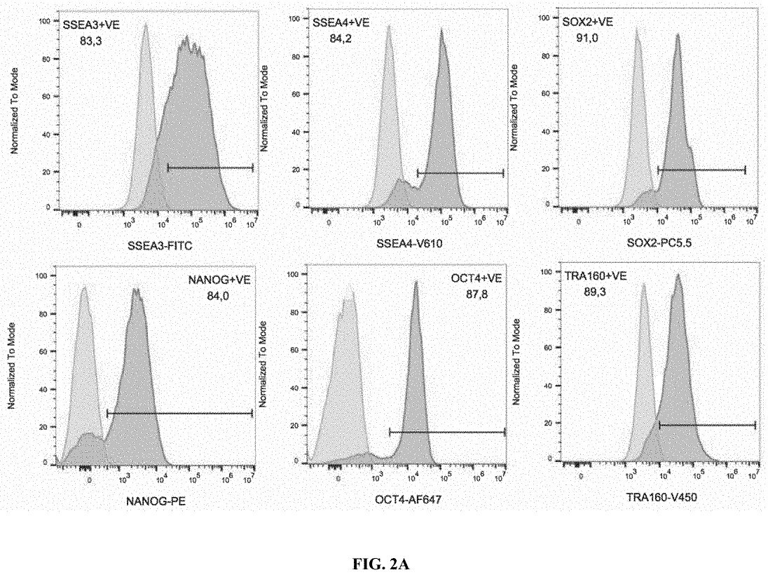

In some embodiments, the alphavirus (e.g., VEEV) RNA construct may have a structure as shown in FIG. 1, comprising, optionally from 5′ to 3′, coding sequences for nsP1, nsP2, nsP3, and nsP4, and a polycistronic expression cassette for expressing a combination of reprogramming factors such as (i) OCT4, KLF4, SOX2, BCL-xL, and c-Myc, (ii) OCT4, SOX2, BCL-xL, and c-MYC, or (ii) OCT4, SOX2, and BCL-xL. In the polycistronic expression cassette, the expression cassette may be under the transcriptional control of a 26S promoter, and/or the coding sequences for the reprogramming factors may be separated by an IRES sequence or a coding sequence for a self-cleaving 2A peptide (non-limiting examples of IRES locations are shown in FIG. 1).

The alphavirus (e.g., VEEV) RNA construct may be produced from a DNA template (e.g., a DNA plasmid construct). By way of example, the RNA construct may be transcribed from a DNA template by using a SP6 (or T7) in vitro transcription kit.

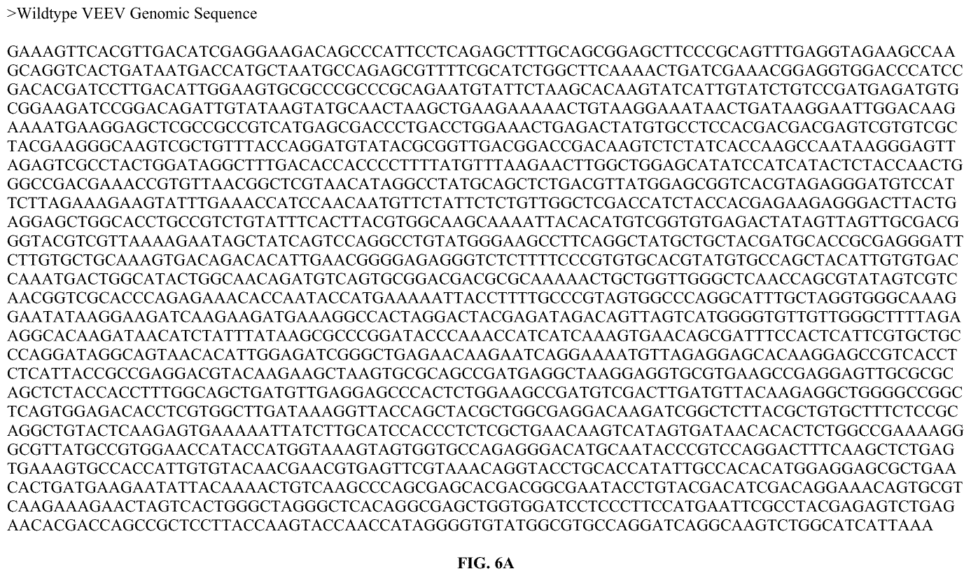

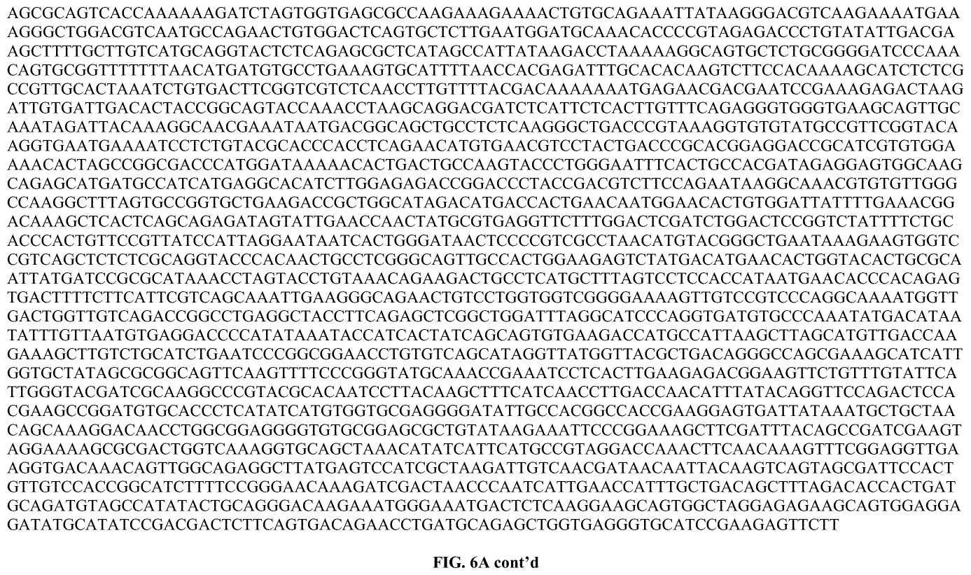

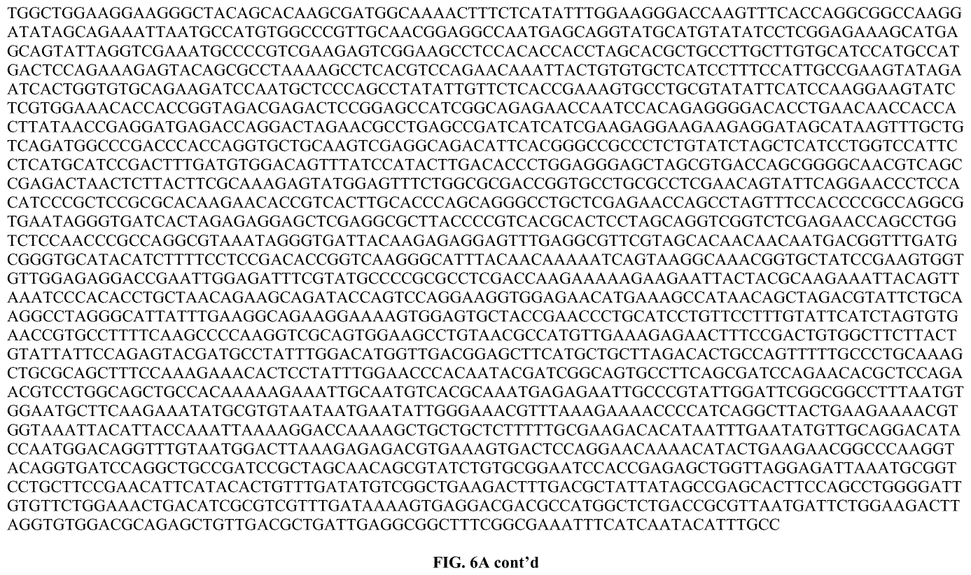

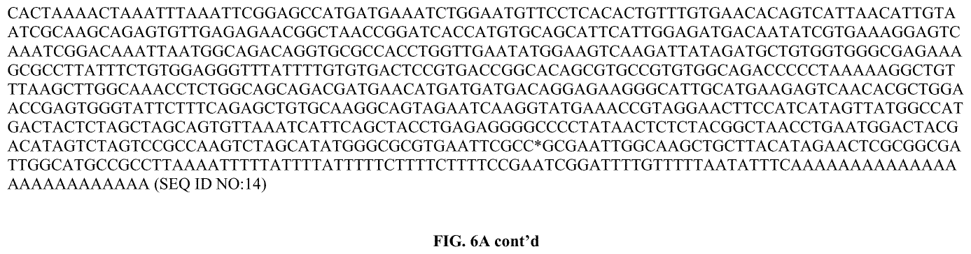

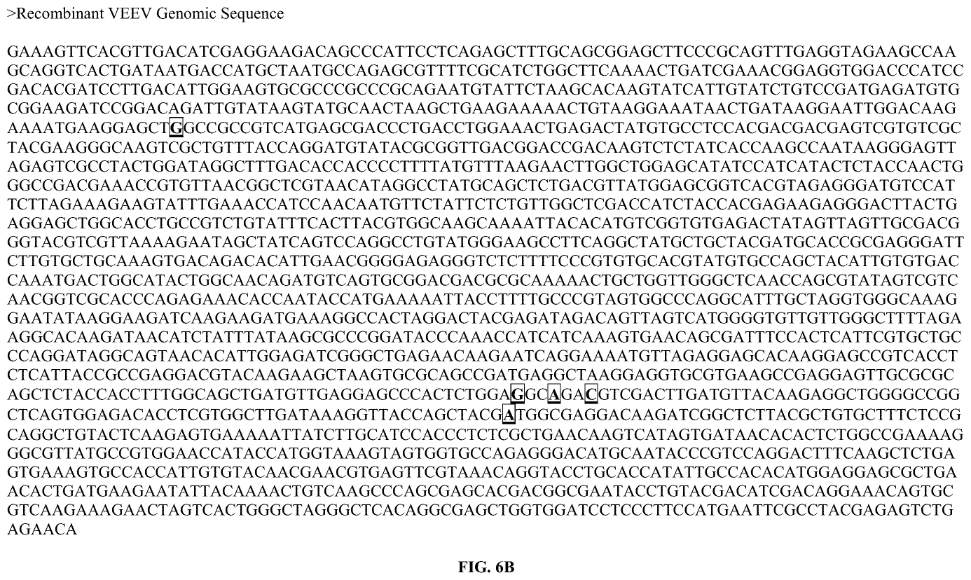

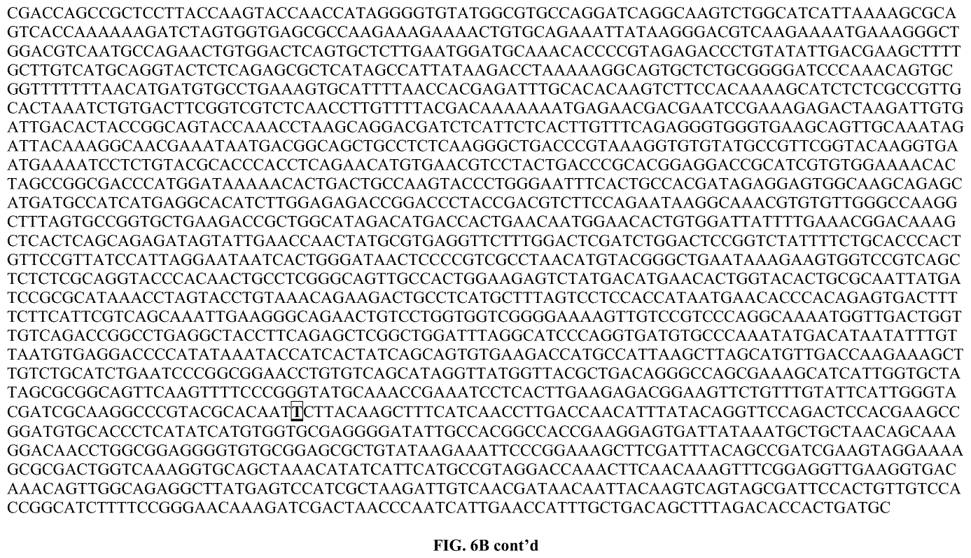

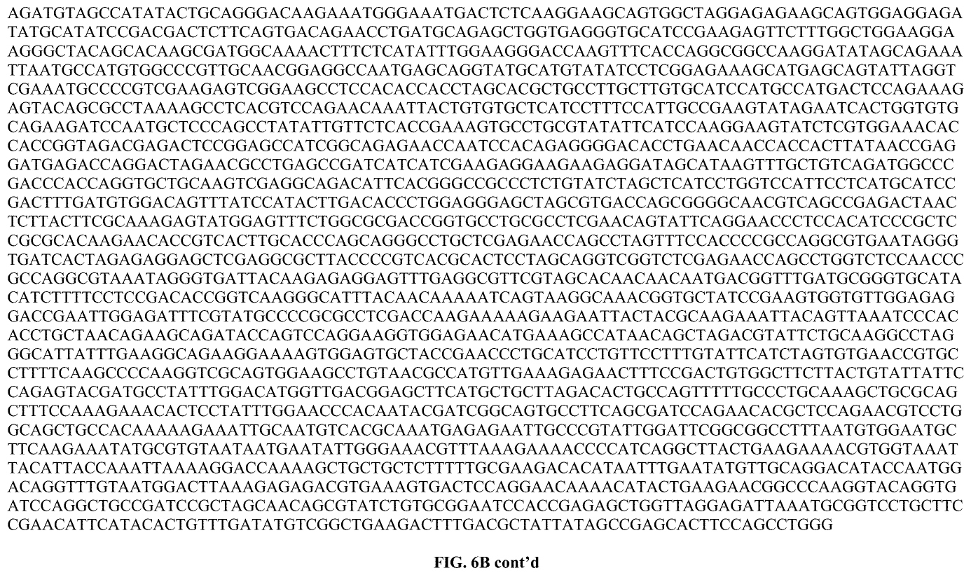

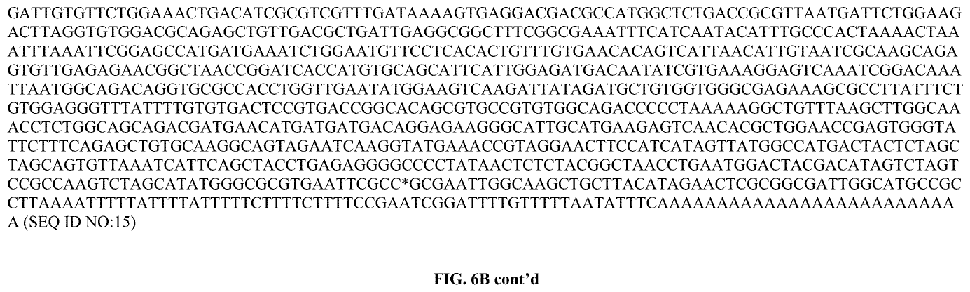

Any strain of VEEV may be used to provide the backbone for the present RNA construct. For example, the TC-83 strain of VEEV may be used. This strain contains a P773S mutation in nsP2 and consequently has reduced cytopathic effect on transduced cells. Other or additional mutations in one or more of the ns proteins may be introduced to improve RNA replication and expression, and/or attenuate immune response to the RNA genome. For example, the VEEV RNA expression construct can comprise one or more (e.g., two, three, four, five, or all six) of those mutations shown in FIG. 6B. Further, in lieu of VEEV, other alphaviruses also may be used to provide the backbone for the reprogramming self-replicating RNA constructs. Nonlimiting examples of other alphaviruses on which the RNA construct may be based are Eastern Equine Encephalitis virus (EEEV), Everglades virus, Mucambo virus, Pixuna virus and Western Equine Encephalitis virus (WEEV), Sindbis virus, Semliki Forest virus, Middelburg virus, Chikungunya virus, O'nyong'nyong virus, Ross River virus, Barmah Forest virus, Getah virus, Sagiyama virus, Bebaru virus, Mayaro virus, Una virus, Aura virus, Whataroa virus, Babanki virus, Kyzylagach virus, Highlands J virus, Fort Morgan virus, Ndumu virus and Buggy Creek virus. The RNA constructs may contain sequences from more than one alphavirus.

II. Generation of iPSCs from Cells of Hematopoietic Lineage Using RNA Constructs

The present methods efficiently reprogram (or termed “dedifferentiate”) blood cells to become induced pluripotent stem cells.

As used herein, the term “pluripotent” or “pluripotency” refers to the capacity of a cell to self-renew and to differentiate into cells of any of the three germ layers: endoderm, mesoderm, or ectoderm. “Pluripotent stem cells” or “PSCs” include, for example, embryonic stem cells derived from the inner cell mass of a blastocyst or derived by somatic cell nuclear transfer, and iPSCs derived from non-pluripotent cells.

The term “induced pluripotent stem cell” or “iPSC” refers to a type of pluripotent stem cell artificially prepared from a non-pluripotent cell, such as an adult somatic cell, partially differentiated cell or terminally differentiated cell, such as a fibroblast, a cell of hematopoietic lineage, a myocyte, a neuron, an epidermal cell, or the like, by introducing or contacting the cell with one or more reprogramming factors.

The starting cell population for PSC induction may be obtained from blood (e.g., peripheral blood) from a patient in need of cell therapy or from a healthy donor. Peripheral blood mononuclear cells (PMBCs) may be isolated by conventional methods and then further fractioned and/or enriched to obtain subsets of cells, e.g., T lymphocytes, B lymphocytes, monocytes, natural killer cells, neutrophils, eosinophils, dendritic cells, and various hematopoietic progenitor cells such as erythroid progenitors, lymphoid progenitors, and myeloid progenitors.

In some embodiments, PMBCs are cultured in a basal medium (e.g., StemSpan™ SFEM II medium; StemCell Technologies) supplemented with erythropoietin (EPO), stem cell factor (SCF) and IL-3 for a period of time (e.g., 3-10 days such as 6, 7, or 8 days) to obtain a cell population enriched for erythroid progenitor cells. The culture medium may be supplemented with, for example, 0.5 to 5 (e.g., 1, 2, 3, or 4) IU/mL EPO, 50 to 200 (e.g., 75, 100, 125, 150, or 175) ng/mL SCF, and 1 to 10 (e.g., 2, 3, 4, 5, 6, 7, 8, or 9) ng/mL IL-3. Other factors that promote erythroid progenitor proliferation may also be used, for example, recombinant human insulin, iron-saturated human transferrin, ferric nitrate, hydrocortisone. See, e.g., Neildez-Nguyen et al., Nat Biotechnol. (2002) 20:467-72; and Filippone et al., PLoS One (2010) 5(3):e9496. Erythroid progenitors can further be isolated from the cell culture by, e.g., fluorescence or magnetic activated cell sorting using reagents (e.g., antibodies) that bind to erythroid progenitor markers such as CD71 and CD36.

In some embodiments, the PBMCs are cultured in the presence of 1 IU/ml EPO, about 100 ng/mL SCF, and about 5 ng/mL IL-3. In some embodiments, the PBMCs are cultured in the presence of 1 IU/ml EPO, about 100 ng/mL SCF, and about 10 ng/mL IL-3. In some embodiments, the PBMCs are cultured in the presence of 1 IU/ml EPO, about 150 ng/mL SCF, and about 5 ng/mL IL-3. In some embodiments, the PBMCs are cultured in the presence of 1 IU/ml EPO, about 150 ng/mL SCF, and about 10 ng/mL IL-3. In some embodiments, the PBMCs are cultured in the presence of 1 IU/ml EPO, about 200 ng/mL SCF, and about 5 ng/mL IL-3. In some embodiments, the PBMCs are cultured in the presence of 1 IU/ml EPO, about 200 ng/mL SCF, and about 10 ng/mL IL-3. In some embodiments, the PBMCs are cultured in the presence of 3 IU/ml EPO, about 100 ng/mL SCF, and about 5 ng/mL IL-3. In some embodiments, the PBMCs are cultured in the presence of 3 IU/ml EPO, about 100 ng/mL SCF, and about 10 ng/mL IL-3. In some embodiments, the PBMCs are cultured in the presence of 3 IU/ml EPO, about 150 ng/mL SCF, and about 5 ng/mL IL-3. In some embodiments, the PBMCs are cultured in the presence of 3 IU/ml EPO, about 150 ng/mL SCF, and about 10 ng/mL IL-3. In some embodiments, the PBMCs are cultured in the presence of 3 IU/ml EPO, about 200 ng/mL SCF, and about 5 ng/mL IL-3. In some embodiments, the PBMCs are cultured in the presence of 3 IU/ml EPO, about 200 ng/mL SCF, and about 10 ng/mL IL-3. In these embodiments, the culturing may be conducted for six, seven, or eight days.

Other subsets of blood cells may also be obtained by fractionation and/or enrichment through cell culture. Markers for specific subsets of blood cells are well-known, such as CD3 for T lymphocytes and CD19 and CD20 for B cells.

The present RNA construct may be introduced into a somatic cell population by a number of techniques including microinjection, electroporation, biolistic particle delivery, lipofection, cationic polymers, and calcium phosphate precipitation. In some embodiments, the present RNA construct is introduced into the somatic cells (e.g., hematopoietic progenitor cells and lymphocytes) through electroporation. While it is known that the use of alphaviruses as vectors may be inhibited by an innate immune response by interferons (IFNs), a type I IFN inhibitor, such as B18R or B19R, may be used to inhibit the cellular antiviral response, thereby enabling desired replicon activity in the cell. In some embodiments, the cells may be treated with the B18R protein prior to electroporation to facilitate alphavirus (e.g., VEEV) delivery and subsequent replication and/or to suppress cellular interferon response in the transfected cells. The electroporated cells may be cultured in the presence of B18R for 2-3 weeks, during which iPSCs emerge and can be harvested. iPSCs may be detected by markers such as TRA-1-60, NANOG, SSEA3, and SSEA4. In some embodiments, culture media such as Opti-MEM® (Thermo Fisher) may be used as an electroporation cell suspension buffer to promote survival of cells post-electroporation. In some embodiments, the RNA construct may be packaged into an alphavirus virion and the virion is used to transduce cells that are to be reprogrammed.

Methods of maintaining iPSCs are well known in the art, and many of such methods are similar to methods of maintaining embryonic stem cells. See, e.g., Thomson et al., Science (1998) 282(5391):1145-7; Hovatta et al., Human Reprod. (2003) 18(7):1404-09; Ludwig et al., Nature Methods (2006) 3:637-46; Kennedy et al., Blood (2007) 109:2679-87; Chen et al., Nature Methods (2011) 8:424-9; and Wang et al., Stem Cell Res. (2013) 11(3):1103-16. The iPSCs may also be cryopreserved prior to use.

II. Differentiation of iPSCs into Target Cell Types

iPSCs are the starting point for the potential generation of large numbers of a specific cell type that can be delivered for regenerative medicine in patients with many different diseases. Differentiation, in the context of iPSC, is the process of lineage specification using cell specific protocols, starting with an iPSC. The iPSCs obtained by the present methods can be differentiated into a cell type of interest for cell therapy, including cells in the endoderm, ectoderm and mesoderm lineages. In some embodiments, the iPSCs may have first been genetically engineered (e.g., to produce a functional protein that is defective in a patient, to produce a therapeutic protein, to include a suicide switch, or to evade immune detection, thereby supporting allogeneic applications) prior to differentiation into a cell type of interest. Methods for inducing differentiation of iPSCs into cells of various lineages and expansion thereof are well known in the art. Non-limiting examples of differentiated cell types are described below.

A. Immune Cells

The iPSCs, optionally having been genetically modified, may be differentiated into immune cells such as lymphoid cells (e.g., T cells, B cells, and NK cells), myeloid cells (e.g., granulocytes, monocytes/macrophages, and tissue-resident macrophages such as microglia), and dendritic cells (e.g., myeloid dendritic cells and plasmacytoid dendritic cells). In some embodiments, the genetically modified cells are T cells expressing a chimeric antigen receptor (CAR) or CAR T cells. The genetically modified immune cells may also express an immunoregulatory transgene such as HLA-G or HLA-E.

For example, methods for inducing differentiation of PSCs into dendritic cells are described in Slukvin et al., J Imm. (2006) 176:2924-32; Su et al., Clin Cancer Res. (2008) 14(19):6207-17; and Tseng et al., Regen Med. (2009) 4(4):513-26. Methods for inducing PSCs into hematopoietic progenitor cells, cells of myeloid lineage, and T lymphocytes are described in, e.g., Kennedy et al., Cell Rep. (2012) 2:1722-35. Methods for inducing PSCs into macrophages are described in van Wilgenburg et al., PLoS One (2013) 8(8):e71098.

The immune cells, such as immunosuppressive immune cells (e.g., regulatory T cells and immunosuppressive macrophages), can be transplanted into a patient having an autoimmune disease, including, without limitation, rheumatoid arthritis, multiple sclerosis, chronic lymphocytic thyroiditis, insulin-dependent diabetes mellitus, myasthenia gravis, chronic ulcerative colitis, ulcerative colitis, Crohn's disease, inflammatory bowel disease, Goodpasture's syndrome, systemic lupus erythematosus, systemic vasculitis, scleroderma, autoimmune hemolytic anemia, and autoimmune thyroid disease. The immune cell-based therapies may also be used in treating graft rejection in transplantation, including treatment of symptoms related to transplantation, such as fibrosis.

B. Neural Cells

The iPSCs, optionally having been genetically modified, may be differentiated into neural cells, including, without limitation, neurons and neuron precursor cells irrespective of any specific neuronal subtype (e.g., dopaminergic neurons, enteric neurons, interneurons, and cortical neurons); glial cells and glial precursor cells irrespective of any specific glial subtype (e.g., oligodendrocytes, astrocytes, dedicated oligodendrocyte precursor cells, and bipotent glial precursors, which may give rise to astrocytes and oligodendrocytes); and microglia and microglia precursor cells. Spinal or oculomotor neurons, enteric neurons, Placode-derived cells, Schwann cells, and trigeminal or sensory neurons are also contemplated

The neural cells can be transplanted into, including, without limitation, a patient having a neurodegenerative disease. Examples of neurodegenerative diseases are Parkinson's disease, Alzheimer's disease, dementia, epilepsy, Lewy body syndrome, Huntington's disease, spinal muscular atrophy, Friedreich's ataxia, amyotrophic lateral sclerosis, Batten disease, and multiple system atrophy, leukodystrophies, transverse myelitis, neuromyelitis optica, lysosomal storage disorders (e.g., Hurler syndrome, Fabry disease, Gaucher disease, Sly syndrome, GM1 and GM2 gangliosidosis, Hunter syndrome, Niemann-Pick disease, Sanfilippo syndrome), tauopathies, among others.

For many of these diseases, the iPSCs may be first directed to adopt a primitive neural cell fate through dual SMAD inhibition (Chambers et al., Nat Biotechnol. (2009) 27(3):275-80). Primitive neural cells adopt anterior characteristics, so the absence of additional signals will provide anterior/forebrain cortical cells. Caudalizing signals can be blocked to prevent paracrine signals that might otherwise generate cultures with more posterior character (for example, XAV939 can block WNT and SU5402 can block FGF signals). Dorsal cortical neurons can be made by blocking SHH activation, while ventral cortical neurons can be made through SHH activation. More caudal cell types, such as serotonergic neurons or spinal motor neurons can be made by caudalizing cultures through the addition of FGF and/or WNT signals. For some cell types, retinoic acid (another caudalizing agent) may be added to posteriorize cultures. The production of glial cell types may generally follow the same patterning of primitive neural cells before extended culture in FGF2 and/or EGF containing medium. PNS cell types may follow the same general principles but with a timely WNT signal early in the differentiation process.

The neural cells may be introduced into the patient through a cannula placed into the damaged tissue in question. A cell preparation may be placed into supportive medium and loaded into a syringe or pipette-like device that can accurately deliver the preparation. The cannula may then be placed into a patient's nervous system, usually using stereotactic methods to precisely target delivery. Cells can then be expelled into the tissue at a rate that is compatible.

C. Cardiovascular Cells

The iPSCs, optionally having been genetically modified, may be differentiated into cells in the cardiovascular system, such as cardiomyocytes including specific cardiomyocyte subtypes (e.g., ventricular or atrial), cardiac fibroblasts, cardiac smooth muscle cells, cardiac epicardium cells, cardiac endocardium cells, cardiac endothelial cells, Purkinje fibers, and nodal and pacemaker cells. Numerous methods exist for differentiating iPSCs into cardiomyocytes, for example as shown in Kattman et al., Cell Stem Cell (2011) 8(2):228-40; Lian et al., PNAS (2012) 109:e1848-57; Lee et al., Cell Stem Cell (2017) 21:179-94, and as shown in WO 2016/131137, WO 2018/098597, and U.S. Pat. No. 9,453,201. Any suitable method in the art can be used with the methods herein to obtain PSC-derived cardiomyocytes.

In some embodiments, the iPSCs are incubated in one or more cardiac differentiation media. For example, the media may contain varying concentrations of bone-morphogenetic protein (BMP, such as BMP4) and activin (such as activin A). Titration of differentiation factor concentration may be performed to determine the optimal concentration necessary for achieving desired cardiomyocyte differentiation.

In some embodiments, the differentiated cardiomyocytes express one or more of cardiac troponin T (cTnT), and/or myosin light chain 2v (MLC2v). In some embodiments, the immature cardiomyocytes express one or more of troponin T, cardiac troponin I, alpha actinin and/or beta-myosin heavy chain.

D. Cells in the Metabolic System

The iPSCs, optionally having been genetically modified, may be differentiated into cells involved with the human metabolic system. For example, the cells may be cells of the gastrointestinal system (e.g., hepatocytes, cholangiocytes, and pancreatic beta cells), cells of the hematopoietic system, and cells of the central nervous system (e.g., pituitary hormone-releasing cells). By way of example, to generate pituitary hormone-releasing cells, iPSCs are cultured with BMP4 and SB431542 (which block activin signaling) before the addition of SHH/FGF8 and FGF10; cells are then subjected only to SHH/FGF8 and FGF10 for an extended period before FGF8 or BMP (or both) to induce the cells to become specific hormone-releasing cells. See, e.g., Zimmer et al., Stem Cell Reports (2016) 6:858-72.

E. Cells in the Ocular System

The iPSCs, optionally having been genetically modified, may be differentiated into cells in the ocular system. For example, the cells may be retinal progenitor cells, retinal pigment epithelial (RPE) progenitor cells, RPE cells, neural retinal progenitor cells, photoreceptor progenitor cells, photoreceptor cells, bipolar cells, horizontal cells, ganglion cells, amacrine cells, Mueller glia cells, cone cells, or rod cells. Methods of differentiating iPSCs into RPE cells are described in, e.g., WO 2017/044483. Methods for isolating RPE cells are described in e.g., WO 2017/044488. Methods for differentiating iPSCs into neural retinal progenitor cells are described in WO 2019/204817. Methods for identifying and isolating retinal progenitor cells and RPE cells are described in e.g., WO 2011/028524.

### IV. Pharmaceutical Compositions and Use

The iPSC-derived cells described herein may be provided in a pharmaceutical composition containing the cells and a pharmaceutically acceptable carrier. The pharmaceutically acceptable carrier may be cell culture medium that optionally does not contain any animal-derived component. For storage and transportation, the cells may be cryopreserved at <−70° C. (e.g., on dry ice or in liquid nitrogen). Prior to use, the cells may be thawed, and diluted in a sterile cell medium that is supportive of the cell type of interest.

The cells may be administered into the patient systemically (e.g., through intravenous injection or infusion), or locally (e.g., through direct injection to a local tissue, e.g., the heart, the brain, and a site of damaged tissue). Various methods are known in the art for administering cells into a patient's tissue or organs, including, without limitation, intracoronary administration, intramyocardial administration, transendocardial administration, or intracranial administration.

A therapeutically effective number of iPSC-derived cells are administered to the patient. As used herein, the term “therapeutically effective” refers to a number of cells or amount of pharmaceutical composition that is sufficient, when administered to a human subject suffering from or susceptible to a disease, disorder, and/or condition, to treat, prevent, and/or delay the onset or progression of the symptom(s) of the disease, disorder, and/or condition. It will be appreciated by those of ordinary skill in the art that a therapeutically effective amount is typically administered via a dosing regimen comprising at least one-unit dose.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Generally, nomenclature used in connection with, and techniques of, cell and tissue culture, molecular biology, immunology, microbiology, genetics, analytical chemistry, synthetic organic chemistry, medicinal and pharmaceutical chemistry, and protein and nucleic acid chemistry and hybridization described herein are those well-known and commonly used in the art. Enzymatic reactions and purification techniques are performed according to manufacturer's specifications, as commonly accomplished in the art or as described herein. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety. Although a number of documents are cited herein, this citation does not constitute an admission that any of these documents forms part of the common general knowledge in the art.

In order that this invention may be better understood, the following examples are set forth. These examples are for purposes of illustration only and are not to be construed as limiting the scope of the invention in any manner.

### EXAMPLES

### Example 1: VEEV Design and RNA Synthesis

This example describes the design of polycistronic VEEV RNA constructs and their synthesis. The constructs used in the following studies were based on the VEEV RNA backbone sequence described in Yoshioka, 2013, supra. That backbone sequence encodes the four non-structural proteins of VEEV. The subgenomic sequence was modified to express various combinations of reprogramming nuclear factors (FIG. 1). RNA construct OKS-iBM encodes OCT4, KLF4, SOX2, BCL-xL, and c-MYC. RNA construct OKS-iGM encodes OCT4, KLF4, SOX2, GLIS1, and c-MYC. RNA construct OKS-iG encodes OCT4, KLF4, SOX2, and GLIS1. RNA construct OSB encodes OCT4, SOX2, and BCL-xL. RNA construct OS-iB encodes OCT4, SOX2, and BCL-xL. RNA construct OS-iM encodes OCT4, SOX2, and c-MYC. RNA construct OS-iBM encodes OCT4, SOX2, BCL-xL and c-MYC. Constructs whose names include “i” contain an IRES sequence immediately downstream of the SOX2 coding sequence. The coding sequences for the various reprogramming factors are separated by coding sequences for a self-cleaving 2A peptide or by an IRES, such that all the reprogramming factors are expressed from the same promoter (Wen et al., Stem Cell Rep. (2016) 6:873-84; Su et al., PLoS ONE (2013) 8:e64496).

The VEEV RNA constructs were enzymatically synthesised from their respective DNA plasmid templates. To optimize RNA synthesis, 5′ capped RNAs were generated using AG Cap analog technology (CleanCap, Trilink).

The backbone sequence of the recombinant VEEV construct is shown in FIG. 6B (SEQ ID NO:15), where the insertion site for expression cassettes is indicated by the asterisk in the sequence.

### Example 2: Reprogramming of Erythroid Progenitors into iPSCs

This example describes an exemplary protocol for reprogramming erythroid progenitor cells into iPSCs. To obtain a cell population enriched for erythroid progenitors (EPs), the PBMCs from human donors were thawed and then cultured for five to ten days (e.g., six days) in a medium supplemented with about 3 IU/mL EPO, about 100 ng/mL SCF, and about 5 ng/mL IL-3 (“EP medium”). These growth factors support EP proliferation. See, e.g., Wang et al., Clin Hemorheol Microcirc. (2007) 37(4):291-9. Alternatively, the PBMCs can be cultured as described in Wen et al. (Stem Cell Reports (2016) 6:873-84), e.g., in a culture medium comprising Stemline® II Hematopoietic Stem Cell Expansion Medium (Sigma; S0192) supplemented with 100 ng/ml stem cell factor (Peprotech; 300-07), 10 ng/ml interleukin-3 (Peprotech; AF-200-03), 2 U/ml erythropoietin (Peprotech; 100-64), 20 ng/ml insulin growth factor-1 (Peprotech; 100-11), 1 mM dexamethasone (Sigma; D4902), and 0.2 mM 1-thioglycerol (Sigma; M6145).

More specifically, thawed PBMCs were seeded in the EP medium to achieve a cell density of 2-3×10⁶ cells/mL in tissue culture-treated plates. About one to three quarters of the medium was changed overnight (e.g., 16-24 hrs) after seeding. On day 2, the cells were transferred to a new vessel (ultra-low adherence, non-tissue culture treated) and daily 25-75% medium changes were performed. On day 5, the cells were diluted two-fold by adding additional EP medium. On day 6 (or 7), one half of the culture medium was changed, and a sample of the EP cells was evaluated by flow cytometry for double positivity for CD71 and CD36.

CD71⁺CD36⁺ EP cells were then incubated with an interferon suppressor (e.g., recombinant B18R protein) for 20 mins. The cells were centrifuged, washed with DPBS, and then resuspended in Opti-MEM™ (Thermo Fisher Scientific) at about 2×10⁷ cells/mL. For every 120 μL of electroporation reaction, 4 μg of VEEV reprogramming RNA was transferred into chilled 1.5 mL microtubes. The cells were then electroporated and plated in B18R-supplemented EP media and fed-batch for 2 days. The plates were coated with substrate such as vitronectin or laminin. On days 3 to 6 post-transfection, cells were fed-batch with B18R-supplemented Essential 7 or ReproTeSR™ medium. Starting on day 7 post-transfection, the cultures underwent daily complete medium changes. Colonies of iPSCs emerged around day 10 post-transfection, and were ready to be picked between days 15-20. Picked colonies were expanded in the absence of B18R and then cryopreserved (referred to as VEE-EP-iPSCs herein).

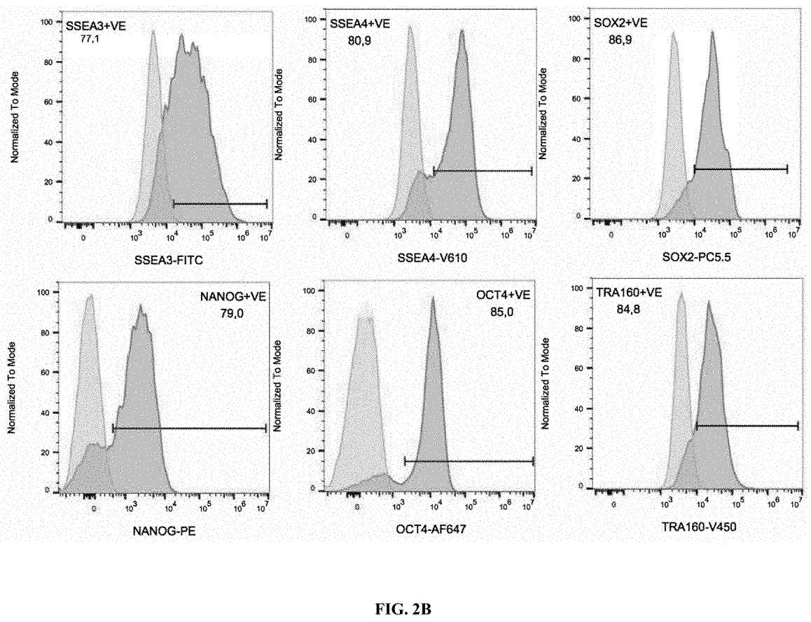

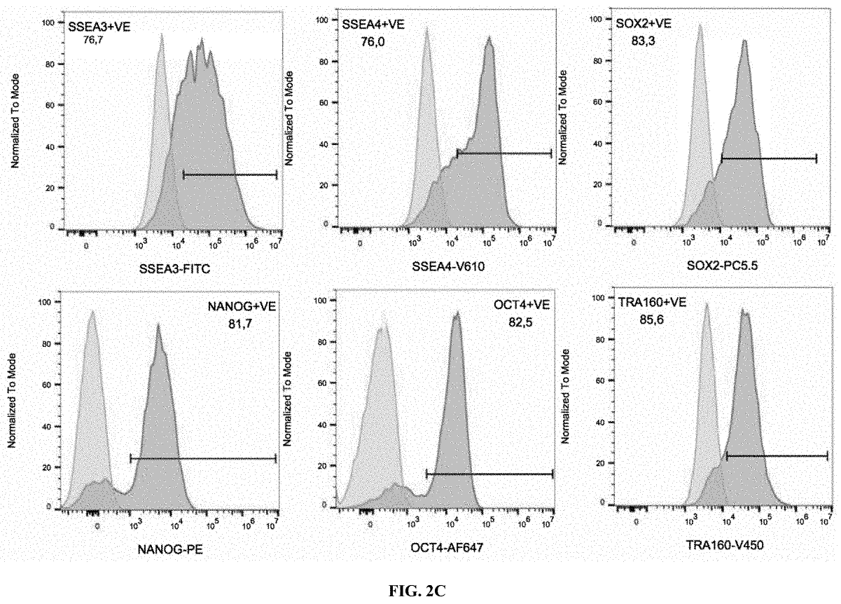

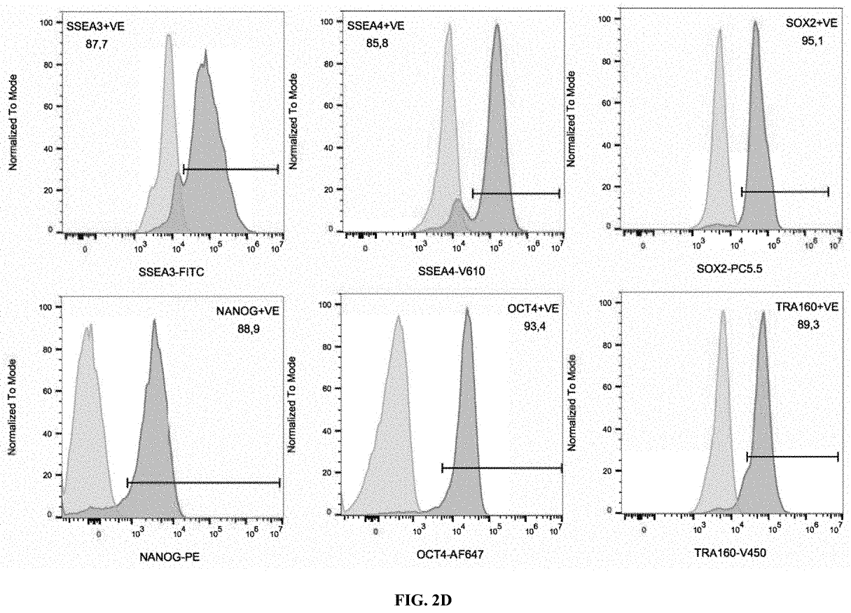

As shown in FIG. 2A-C, VEE-EP-iPSCs that were generated with the three VEEV RNA constructs expressed nuclear (NANOG) and surface markers (TRA-1-60, SSEA-3 and SSEA-4) associated with undifferentiated pluripotent cells. These cells also possessed a normal karyotype.

The VEE-EP-iPSCs were profiled by next-generation sequencing to assess the acquisition of genetic variants in more than 500 cancer-associated genes. When genetic sequences of the more than 500 genes in VEE-EP-iPSC lines were compared to the starting population of donor PBMCs, no differences in sequence were observed. These data demonstrate that genetic variants were not acquired during reprogramming.

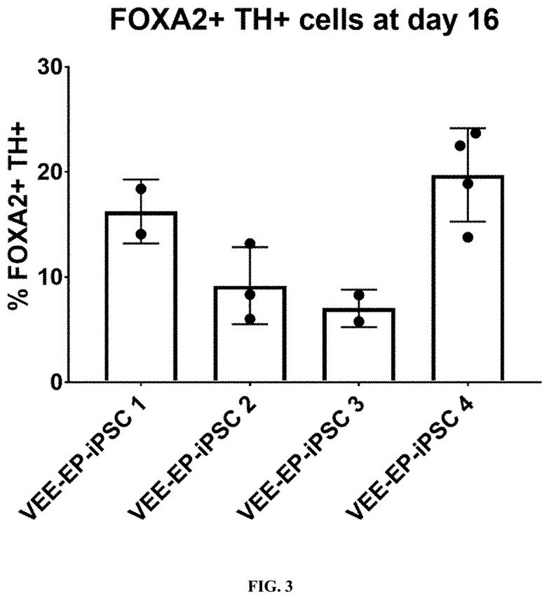

All VEE-EP-iPSC cell lines demonstrated the ability to differentiate into TH⁺ dopaminergic neurons that represent ectoderm (FIG. 3). To direct differentiation of VEE-EP-iPSCs towards dopaminergic neurons, we first induced the iPSCs toward the neurectoderm lineage by blocking TGF-β and BMP signaling from day 0 to day 7. We simultaneously patterned the cells to a floor plate fate via recombinant C25II SHH proteins from day 0 to day 7, and fine-tuned the cells' midbrain identity using the WNT-agonist small molecule CHIR-99021 from day 0 to day 12. Following these steps, we matured the progenitors to a neuronal fate using neurotrophic factors from day 10 to 16 and the small molecule γ-secretase inhibitor DAPT from day 12 to day 16. The day 16 differentiated cells were matured for 5 days in vitro and TH⁺FOXA2⁺ dopamine neurons were quantified by flow cytometry.

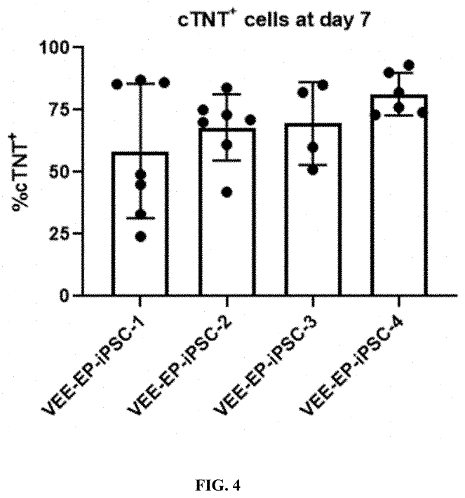

All VEE-EP-iPSC cell lines also were able to differentiate into cardiac troponin (cTNT) positive cardiomyocytes (FIG. 4) that represent mesoderm differentiation. The VEE-EP-iPSC lines were differentiated towards the cardiac lineage with stage-specific modulation of WNT signaling through the use of WNT agonist CHIR-99021 and WNT antagonist endo-IWR1. Cardiomyocytes were quantified by flow cytometry for cardiac troponin (cTNT) staining.

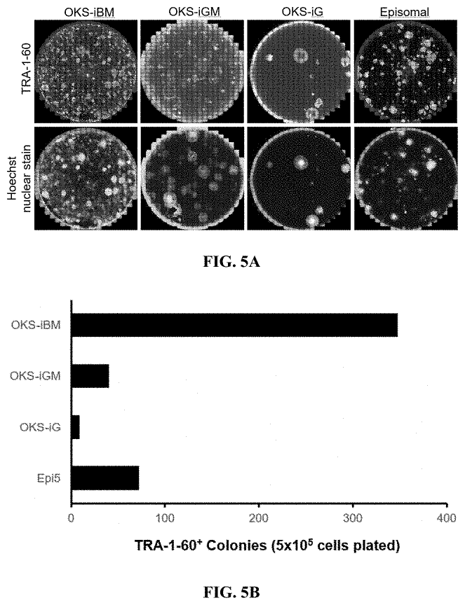

To determine the reprogramming efficiencies of VEEV RNA constructs containing different transcription factor combinations, erythroid progenitors were expanded from PBMCs and electroporated with reprogramming RNA constructs. TRA-1-60 positive colonies were quantified 17 days (for OKS-iBM and episomal constructs) or 25 days (for OKS-iGM and OKS-iG constructs) post-electroporation. The efficiency of reprogramming was determined as the number of cell-based colonies expressing the PSC marker TRA-1-60 (FIG. 5A). The efficiency of VEEV RNA mediated EP reprogramming significantly increased when a BCL-xL coding sequence was included in the VEEV construct (FIG. 5B). The VEEV OKS-iBM construct, which contained a BCL-xL coding sequence, demonstrated four-fold higher EP reprogramming efficiency than an episomal-based control that contains the traditional reprogramming factors OCT4, SOX2, KLF4, L-MYC, LIN28, and p53 dominant negative (FIG. 5B—iBM/Epi5). The OS-iBM construct also was active and formed iPSC colonies (data not shown).

When Su et al. (2013, supra) included BCL-xL as a fifth reprogramming factor (Episomal OS+MK+B combination), they observed an approximately 8-fold increase in reprogramming efficiency, in comparison to OS+MK (OCT4, SOX2, c-MYC and KLF4) combination. However, when Yoshioka and Dowdy (2017, supra) evaluated reprogramming factor combinations, the inclusion of GLIS1 (VEE-OKS-iGM) boosted reprogramming efficiency by approximately 20-fold in comparison to the four-factor combination (VEE-OKS-iM). Thus, it was unexpected that replacing GLIS1 with BCL-xL (VEE-OKS-iBM) increased the reprogramming efficiency of erythroid progenitors by a further 8-fold (FIG. 5B—iBM/iGM). This surprising and dramatic increase in reprogramming efficiency transforms low-efficiency erythroid reprogramming into a robust approach. The more robust efficiency allows consistent reprogramming across different blood samples and is critical to the feasibility of EP reprogramming for clinical use.

### Example 3: Reprogramming of T Cells into iPSCs

This example describes a protocol for reprogramming T lymphocytes into iPSCs. Purified CD3⁺ T cells (pan-T cells) were obtained by negative immuno-selection and immuno-phenotyping of peripheral blood from two independent donors (AllCells). Pan-T cells from both donors were thawed and maintained in a T cell complete medium.supplemented with CTS™ GlutaMAX™ and 100 IU/mL IL-Prior to electroporation, pan-T cells were treated with 0.2 μg/mL recombinant B18R protein for 30 min, washed with cold phosphate-buffered saline and resuspended.).

Cells were electroporated, plated and incubated on Ultra-Low Attachment plates (Corning) for 24 hrs in the above T cell medium supplemented with 0.2 μg/mL of B18R, CTS™ GlutaMAX™, and 100 IU/mL IL-2. Next, the cells were re-plated onto tissue culture plates coated with LN521, freshly supplemented with 0.2 μg/mL B18R. Between days 3 and 6 post electroporation, reprogramming cultures were fed-batch with the StemFit Basic03 medium (Ajinomoto), which was supplemented daily with 0.2 μg/mL of B18R. Starting on day 7 post-transfection, the cultures underwent daily complete medium change with B18R-supplemented StemFit Basic03 medium. Colonies of iPSCs emerged around day 10 post-transfection, and were ready to be picked between days 15-20. Picked colonies were expanded with StemFit Basic03 medium in the absence of B18R and then cryopreserved (referred to as VEE-T-iPSCs herein).

In conclusion, we have demonstrated that, in addition to erythroid progenitors, CD3⁺ T-cells can be reprogrammed by electroporation with VEE-OKS-iBM RNA. Two different donor T-cell lots were reprogrammed to establish iPSC lines, with a reprogramming efficiency of 0.005% averaged between the two donors. This is a sufficient cell line derivation rate as it would result in 50 colonies per million transfected cells.

### LIST OF SEQUENCES

Exemplary sequences of the present disclosure are provided in the table below (SEQ: SEQ ID NO).

TABLE 1

| SEQ | Description |
|---|---|
| 1 | Human BCL-xL amino acid sequence |
| 2 | An exemplary human BCL-xL amino acid sequence expressed |
| | herein |
| 3 | Human OCT4 amino acid sequence |
| 4 | An exemplary human OCT4 amino acid sequence expressed herein |
| 5 | Human KLF4 amino acid sequence |
| 6 | An exemplary human KLF4 amino acid sequence expressed herein |
| 7 | Human SOX2 amino acid sequence |
| 8 | An exemplary human SOX2 amino acid sequence expressed herein |
| 9 | Human c-MYC amino acid sequence |
| 10 | An exemplary human c-MYC amino acid sequence expressed |
| | herein |
| 11 | Human GLIS1 amino acid sequence |
| 12 | Human NANOG amino acid sequence |
| 13 | Human LIN28B amino acid sequence |
| 14 | Nucleotide sequence of wildtype VEEV genome |
| 15 | Nucleotide sequence of a recombinant VEEV RNA expression |
| | vector |

1. A method of obtaining a population of induced pluripotent stem cells (iPSCs) from starting cells of a hematopoietic lineage, comprising:
introducing to the starting cells an alphavirus RNA expression construct encoding BCL-xL and one or more additional reprogramming factors selected from an OCT family protein, a KLF family protein, a MYC family protein, a SOX family protein, a LIN28 protein, a NANOG protein, and a p53 dominant negative protein, and
culturing the starting cells to allow the expression of BCL-xL and the one or more additional reprogramming factors, thereby inducing the starting cells and their progeny to reprogram into iPSCs.

2. The method of claim 1, wherein the starting cells are hematopoietic stem cells, erythroid progenitor cells, lymphoid progenitor cells, peripheral blood mononuclear cells, T lymphocytes, B lymphocytes, macrophages, monocytes, neutrophils, eosinophils, or dendritic cells of human origin.

3. The method of claim 2, wherein the starting cells are erythroid progenitor cells obtained by culturing peripheral blood mononuclear cells (PBMCs) in the presence of erythropoietin, stem cell factor, and IL-3, optionally for five to ten or six to seven days.

4. The method of claim 3 wherein the PBMCs are cultured in the presence of 0.5-5 IU/ml erythropoietin, 50-200 ng/mL stem cell factor, and 1-10 ng/mL IL-3.

5. The method of claim 1, wherein the RNA expression construct is introduced to the starting cells through electroporation.

6-7. (canceled)

8. A population of induced pluripotent stem cells (iPSCs) obtained from starting cells of a hematopoietic lineage that are transfected with an alphavirus RNA expression construct encoding BCL-xL and one or more additional reprogramming factors selected from an Oct family protein, a KLF family protein, a Myc family protein, a SOX family protein, a LIN28 protein, a NANOG protein, and a p53 dominant negative protein.

9. The population of iPSCs of claim 8, wherein the starting cells are hematopoietic stem cells, erythroid progenitor cells, lymphoid progenitor cells, peripheral blood mononuclear cells, T lymphocytes, B lymphocytes, macrophages, monocytes, neutrophils, eosinophils, or dendritic cells of human origin.

10. The population of iPSCs of claim 9, wherein the starting cells are erythroid progenitor cells obtained by culturing peripheral blood mononuclear cells (PBMCs) in the presence of EPO, SCF, and IL-3, optionally for five to ten or six to seven days.

11. An alphavirus RNA expression construct encoding BCL-xL and one or more additional reprogramming factors selected from an OCT family protein, a KLF family protein, a MYC family protein, a SOX family protein, a LIN28 protein, a NANOG protein, and a p53 dominant negative protein.

12. A DNA vector comprising a coding sequence for the alphavirus RNA expression construct of claim 11.

13. A host cell comprising the alphavirus RNA expression construct of claim 11, optionally wherein the host cell is a human cell.

14. (canceled)

15. The method of claim 1, wherein the alphavirus RNA expression construct is a Venezuelan equine encephalitis virus (VEEV) RNA expression construct and comprises VEEV nsP1, nsP2, nsP3, and nsP4 genes,
optionally wherein the VEEV RNA expression construct contains one or more, optionally two or more, three or more, four or more, five or more, or six or more mutations from the corresponding region(s) of wildtype VEEV genome.

16. The method of claim 1, wherein
the OCT family protein is OCT4, optionally a human OCT4,
the KLF family protein is KLF4, optionally a human KLF4,
the SOX family protein is SOX2, optionally a human SOX2,
the LIN28 protein is LIN28B, optionally a human LIN28B, and/or
the MYC family protein is c-MYC, optionally a human c-MYC.

17. The method of claim 1, wherein the alphavirus RNA expression construct directs expression of:
a BCL-xL protein comprising SEQ ID NO:1 or an amino acid sequence at least 95% identical thereto,
an OCT4 protein comprising SEQ ID NO:3 or an amino acid sequence at least 95% identical thereto,
a KLF4 protein comprising SEQ ID NO:5 or an amino acid sequence at least 95% identical thereto,
a SOX2 protein comprising SEQ ID NO:7 or an amino acid sequence at least 95% identical thereto, and/or
a c-MYC protein comprising SEQ ID NO:9 or an amino acid sequence at least 95% identical thereto.

18. The method of claim 1, wherein the alphavirus RNA expression construct directs expression of an OCT family protein, a SOX family protein, BCL-xL, and a MYC family protein, and optionally a KLF family protein.

19. The method of claim 1, wherein the coding sequences for BCL-xL and the one or more additional reprogramming factors are separated by a coding sequence for a 2A peptide or an internal ribosome entry site (IRES).

20. The method of claim 1, wherein the coding sequences for BCL-xL and the one or more additional reprogramming factors are under the transcriptional control of a common promoter, optionally a 26S promoter.

21. A method of obtaining a differentiated cell in vitro, comprising culturing the population of iPSCs of claim 8 in the presence of differentiation-promoting agents.

22. A differentiated cell obtained by the method of claim 21.

23. (canceled)

24. The method of claim 21, wherein the differentiated cell is
a human immune cell, optionally selected from a T cell, a T cell expressing a chimeric antigen receptor (CAR), a suppressive T cell, a myeloid cell, a dendritic cell, and an immunosuppressive macrophage;
a cell in the human nervous system, optionally selected from dopaminergic neuron, a microglial cell, an oligodendrocyte, an astrocyte, a cortical neuron, a spinal or oculomotor neuron, an enteric neuron, a Placode-derived cell, a Schwann cell, and a trigeminal or sensory neuron;
a cell in the human cardiovascular system, optionally selected from a cardiomyocyte, an endothelial cell, and a nodal cell;
a cell in the human metabolic system, optionally selected from a hepatocyte, a cholangiocyte, and a pancreatic beta cell; or
a cell in the human ocular system, optionally selected from a retinal pigment epithelial cell, a photoreceptor cone cell, a photoreceptor rod cell, a bipolar cell, or a ganglion cell.

25. The method of claim 21, wherein the differentiated cell is of ectoderm lineage, optionally wherein the differentiated cell is a neuron.

26. The method of claim 21, wherein the differentiated cell is of mesoderm lineage, optionally wherein the differentiated cell is a cardiomyocyte.

27. A pharmaceutical composition comprising the differentiated cell of claim 22 and a pharmaceutically acceptable carrier.

28. A method of treating a patient in need thereof, comprising administering the differentiated cell of claim 22 to the patient.

29-30. (canceled)
